(19) United States
(12) Patent Application Publication
(10) Pub. No.: US 2022/0257503 A1
(43) Pub. Date: Aug. 18, 2022

(54) Surface Modified Gas Vesicles, Preparation and Applications thereof

(71) Applicant: THE HONG KONG POLYTECHNIC UNIVERSITY, Hong Kong (CN)

(72) Inventors: Lei SUN, Hong Kong (CN); Lin SONG, Hong Kong (CN); Guohao WANG, Hong Kong (CN); Xuandi HOU, Hong Kong (CN)

(21) Appl. No.: 17/595,855

(22) PCT Filed: May 29, 2020
(86) PCT No.: PCT/CN2020/093171

§ 371 Date: Apr. 26, 2022

Related U.S. Application Data
(60) Provisional application No. 62/853,747, filed on May 29, 2019.
(60) Provisional application No. 62/853,739, filed on May 29, 2019.

(51) Int. Cl. A61K 9/00; A61K 45/06; A61K 9/50

(52) U.S. Cl. CPC A61K 9/0009; A61K 9/50; A61K 45/06

(57) ABSTRACT

The present application provides surface-modified novel targeting GVs (such as PH-GVs), lipid GVs and lipid targeting GVs, preparation methods therefor, and applications thereof in tumor diagnosis, imaging and treatment.

### CROSS-REFERENCE TO RELATED APPLICATIONS

The present application is a National Phase entry application of PCT International Application No. PCT/CN2020/093171, filed May 29, 2020, which claims priorities from U.S. Provisional Patent Application No. 62/853,739 filed on May 29, 2019; and U.S. Provisional Patent Application No. 62/853,747 filed on May 29, 2019, all of which are incorporated herein by reference in their entireties.

### TECHNICAL FIELD

The present disclosure relates to surface-modified targeting GVs (such as PH-GVs), lipid GVs and lipid targeting GVs, preparation methods therefor, and applications thereof in tumor diagnosis, imaging and treatment.

### BACKGROUND

Contrast-enhanced ultrasound (CEUS), with gas-filled microbubbles as contrast agents, plays important roles in diagnosis and management of a broad range of diseases. Molecularly-targeted contrast agents, with surface modification by attaching binding ligands to the microbubble shell, facilitate the visualization of overexpressing biomarkers at the molecular level and significantly improve imaging sensitivity and specificity. Since the microbubbles are several micrometers in diameter, they remain exclusively within the vascular compartment. Such property makes them particularly well suited for visualizing molecular markers expressed on the tumor neovasculature.

While the utilization of microbubbles for both imaging and therapy has shown encouraging results, their potential utility in biomedicine has been limited by the large hydrodynamic size (1-8 μm), which hinders their penetration into the surrounding nonvascular tissue after intravenous injection. Additionally, the gas content of these microbubbles diffuses quickly into the surrounding medium, resulting in a very short half-life (<20 min).

Recent studies have confirmed that nanoscale ultrasound contrast agents with a particle size of less than 1000 nm exhibit the following advantages: small molecular weight, strong penetrating power and improved stability. This type of contrast agent includes fluorocarbon emulsion nanodroplets, nanobubbles and nanoparticles. Because of the nanoscale size, they can overcome the above mentioned limitations and cross the leaky, defective vasculature of the tumor while staying in the vasculature of healthy tissue due to the Enhanced Permeability and Retention (EPR) effect. Complementary to diagnostic imaging, these nanoscale ultrasound contrast agents have the potential to work as active drug carriers for localized drug delivery with the capacity to actively trigger drug release with both spatial and temporal specificity due to transient increase in permeability of vasculature and cellular membrane. Despite their attractive benefits, nanoscale ultrasound contrast agents for diagnostic purposes have not experienced great success in clinical translation. Their low gas density and low contrast-to-noise ratio do not allow for high contrast under ultrasound at diagnostic frequencies.

Recently reported gas vesicles (GVs), isolated from buoyant photosynthetic microbes, demonstrated significant potentials as a novel nanoscale contrast agent for ultrasound imaging. GVs produce robust ultrasound contrast across a range of frequencies at picomolar concentrations, exhibit harmonic scattering to enable enhanced detection versus background in vivo. Their nonlinear oscillations generate harmonics of the incident ultrasound that can be specifically detected to further enhance the contrast-to-noise ratio for increased sensitivity and specificity. GVs have a width of 45-250 nm and a length of 200-600 nm, possible to enter the tissue space via the EPR effect. Unlike traditional ultrasound contrast agents, which trap preloaded gas in an unstable configuration, GVs' 2 nm-thick protein shells exclude water but permit gas to freely diffuse in and out from the surrounding media, making them physically stable despite their nanometer size. Furthermore, the prior art has demonstrated the genetic engineering of GVs could enable the design of GV-based contrast agents with new mechanical, acoustic, surface, and functional properties to enable harmonic, multiplexed, and multimodal ultrasound imaging as well as cell-specific molecular targeting. With the above benefits, GVs are very promising nanoparticles for ultrasound (US) molecular imaging and further therapy of cancers.

However, similar to other nanoparticles, the majority of GVs are up-taken in non-targeted tissues such as liver, spleen, and lung following intravenous administration. Previous finding believes that the reticuloendothelial system (RES) removed 84% of native GVs due to the capture by phagocytic cells and collapsed GVs with the biliary system and no GVs remained in the blood 2 min after their injection. This limits the utility of native GVs for tumor molecular imaging as GVs will not circulate long enough to extravasate into target tissues. In addition, they do not have the ability to be specifically internalized into the target cells, resulting in the release of a significant portion of the contrast agents at the extracellular phase, which would further reduce their imaging and therapeutic efficiency. Thus, to translate GVs to novel US molecular imaging agents, further research efforts should be focused on improving pharmacokinetic properties of GVs.

For a long time, tumor hypoxia has been recognized as a critical issue in oncology. Pathological hypoxia could induce proteomic and genomic changes within the tumor cells as well as the changes of the tumor microenvironment. These could alter the local metabolism of tumor cells and activate adaptive cellular responses that contribute to tumor progression. Moreover, the existence of hypoxia makes solid tumors more resistant to radiotherapy and chemotherapy which lead to the poor survival of patients. Given the role of hypoxia in tumor progression and resistance to cancer therapy, people start to think about whether oxygenating the tumor tissue could prevent cancer growth and development. Tumor oxygenation has been demonstrated to be able to improve and help overcome chemotherapeutic and radio-therapeutic resistance significantly by increasing cellular sensitivity. A few studies also implied that tumor oxygenation is related to reduced cell proliferation, angiogenesis and metastasis, and oxygenation might have tumor inhibitory effects in certain tumor subtypes. The change of hypoxic tumor microenvironment could reduce the expression of HIF-1α and thus weaken the hypoxia-driven pathways. Besides, oxygenation in the tumor has been reported to play a role in epigenetic programming. Several hypoxia-associated and tumor suppressor genes could be reprogrammed after oxygenation. This means oxygenation could not only serve as an adjuvant to cancer treatment but have the potential to reverse the proteomic and genomic changes in tumor site caused by hypoxia.

Given the significant effects of oxygen in tumor progression and resistance to therapy, tumor oxygenation has been considered to be an auxiliary to anticancer therapy. Hyperbaric oxygen (HBO) therapy is a well-acknowledged way for treating hypoxia-related disorders. HBO therapy can effectively elevate the oxygen levels of a tumor site and can improve the outcome of chemotherapy and radiotherapy, still, they face the limitations of high expense, uncomfortable experience for patients etc. Besides, oxygen can't be delivered deep in hypoxic tumors which could compromise clinical effects in certain circumstances. Due to the limitation of HBO therapy, scientists have attempted to develop oxygen carriers that can transport significant amounts of oxygen to tumors. Artificial red blood cell (RBC) substitutes such as perfluorocarbon (PFC) emulsions and acellular hemoglobin-based oxygen carriers (HBOCs) have been developed as the first generation of oxygen carriers. Still, most products failed to achieve adequate circulatory half-lives and maintain physiological tissue oxygenation. Recently, with the development of novel oxygen-containing microparticles, nanoparticles emerged as a suitable carrier to reach the tissue of interest and deliver oxygen efficiently.

For oxygen carriers, lipid-based microbubbles are preferred for medical applications due to their good biocompatibility and biodegradability. Microbubbles are capable of carrying a large quantity of oxygen and can be used in connection with ultrasound for site-controlled oxygen release. Oxygen-filled microbubbles have been demonstrated to be able to change the hypoxic microenvironment in vivo and enhance the outcome for chemotherapy and radiotherapy. Further development of oxygen-filled microbubbles still faces some challenges and limitations. First, oxygen-filled microbubbles may suffer from stability issues such as dissolution and coalescence when they entered the circulation system and this is correlated with greater product loss which could lead to excessive production of reactive oxygen species (ROS). Besides, microbubbles couldn't pass through the vasculature due to their size limitation that may compromise their effects if combined with drug delivery.

Photodynamic therapy (PDT), has emerged as a successful clinical therapeutic modality for cancer treatment which has already been approved by Food and Drug Administration (FDA). PDT relies on the synergistic interaction between photosensitizers and the corresponding light. The two elements are individually non-toxic, but they could initiate chemical reaction and achieve tumor cell apoptosis when working together. Photosensitizers could selectively enter the tumor site through blood circulation and give rise to large quantity of reactive oxygen species (ROS) when excited by the appropriate activating wavelength of light. ROS, especially singlet oxygen radicals, is believed to be responsible for the necrosis and apoptosis of cancer cells. Besides producing ROS to direct kill cancer cells, PDT could also cause immunoreaction against the cancer cells after treatment as well as inhibit tumor growth by cutting off the nutrition supply through cancer-associated vasculature damage. Due to the advantages of well recognized safety, selectivity and repeatability, PDT has been applied not only in the field of dermatology but also used as adjuvant therapy to treat pulmonary, respiratory tract, and urinary tract tumors. During PDT treatment, the availability of oxygen can be a limiting factor for efficacy because ROS production is dependent on the concentration of oxygen. However, cancer tissues often show the situation of hypoxia and this usually limits the effectiveness of PDT. Thus, improving oxygen concentration in the tumor site is a good strategy for enhanced efficacy of PDT. Oxygen-filled microbubbles have been used for enhanced PDT and the efficacy has been approved both in vitro and in vivo. For example, previous studies have proved that the existence of microbubbles could lead to more production of singlet oxygen and enhanced cellular apoptosis during PDT. This means that oxygen-filled microbubbles together with photosensitizers could lead to enhanced cytotoxicity. However, due to the limitation of stability and relatively large size, the application of oxygen-filled microbubbles is compromised.

Sonodynamic therapy (SDT) has emerged as a promising non-invasive therapeutic modality compared to those traditional cancer therapies such as surgery, chemotherapy and radiotherapy in the enduring battle against cancer. SDT is a kind of ultrasound therapy in which sonosensitizers are administered to increase the efficacy of ultrasound's cytotoxicity to tumors while leaving normal tissue undamaged and intact. The concept of SDT is very similar to the well-established photodynamic therapy (PDT) in which laser is used to activate photosensitizers to produce toxicity. Compared to PDT whose application is limited to superficial tumors due to the limited penetration of laser light, SDT has the advantage of being able to treat deep-seated cancer since ultrasound can be focused on a single point deeply within tissues in three dimensions. The effects of SDT on cancer treatment have been demonstrated widely both in vitro and in vivo. The generation of reactive oxygen species (ROS) in the presence of acoustic fields is responsible for the cytotoxic effects of SDT. Cavitation induced by ultrasound is believed to be involved in the interaction of ultrasound and sensitizers to generate ROS. Cavitation is the behavior of gas bubbles in aqueous environments with the interaction of ultrasound. Cavitation includes process of nucleation, growth and the implosive collapse of gas-filled bubbles. Inertial cavitation during which bubbles rapidly expand and violently collapse in a liquid medium is closely associated with excessive production of ROS. Thus, microbubbles have been used to serve as artificial nuclei for enhanced ultrasound triggered inertial cavitation in the therapeutic application of SDT. The addition of microbubbles could decrease cavitation threshold, and consequently enhance ROS production and cytotoxicity. However, the application of microbubbles in SDT therapy is also limited by its poor stability and relatively large size.

Therefore, in order to overcome the problems in the diagnosis and treatment of cancers and some other diseases in the prior art, it is urgent to develop novel GVs with improved pharmacokinetic properties to improve the accuracy of neoplastic diseases diagnosis and therapeutic effects.

### SUMMARY OF THE INVENTION

In order to address the problems existing in the prior art, the present invention provides novel surface-modified targeting GVs (such as PH-GVs), lipid GVs and lipid targeting GVs, preparation methods therefor, and applications thereof.

The first aspect of the present invention provides a surface-modified targeting GV capable of specifically targeting tumor sites. The targeting GV of the present invention is surface-modified with a biocompatible material such as polyethylene glycol (PEG), chitosan, polyurethane, polylactic acid, polyolefin, polysulfone, polycarbonate, polyacrylonitrile and the like, or any combination thereof, and/or with a targeting biomaterial such as hyaluronic acid (HA), RGD peptide, folic acid, galactose, glucose and the like, or any combination thereof. In some embodiments, the PEG used in the present invention has a molecular weight of about 5000 Da. The targeting GV is a GV with surface being modified with PEG and/or HA, preferably a GV surface-modified with PEG and HA (PH-GV).

In some embodiments, the targeting GV of the present invention, such as PH-GV, is cylindrical in shape with a particle size of about 400 nm. The targeting GV has a particle size of about 375-425 nm, and a zeta potential of about −23 to −31 mV as measured by the dynamic light scattering (DLS) method.

The second aspect of the present invention provides a contrast agent or diagnostic agent containing the targeting GV of the first aspect of the present invention, such as PH-GV. In some embodiments, the targeting GV is present in the contrast agent at a concentration of about 250 pM-1 nM.

The third aspect of the present invention provides a diagnostic kit containing the contrast agent or diagnostic agent of the second aspect of the present invention, and optionally an instruction for use.

In some embodiments, the targeting GV of the first aspect of the present invention, the contrast agent or diagnostic agent of the second aspect, and the kit of the third aspect of the present invention can be used to diagnose diseases such as cancers. The targeting GV or the contrast agent can be used to image a tumor to diagnose the presence or absence of a cancer. Therefore, the present invention also provides the use of the targeting GV of the first aspect or the contrast agent or diagnostic agent of the second aspect in the preparation of a diagnostic agent or a diagnostic kit for diagnosing diseases such as cancers. The present invention also provides the use of the targeting GV or the contrast agent in the preparation of a diagnostic reagent or a diagnostic kit for imaging tumors.

The fourth aspect of the present invention provides a diagnostic method, comprising using the targeting GV of the first aspect of the present invention, the contrast agent or diagnostic agent of the second aspect, or the kit of the third aspect to diagnose the presence or absence of a cancer in a subject. In some embodiments, the diagnosis includes administering the targeting GV, the contrast agent or diagnostic agent to the subject, or applying the kit to the subject. The diagnostic method further comprises applying ultrasound imaging to the subject to determine the presence or absence of the cancer.

The targeting GV of the present invention can also be used as a drug carrier. Therefore, the fifth aspect of the present invention provides a drug carrier containing or consisting of the targeting GV of the first aspect of the present invention, such as PH-GV. In some embodiments, the drug carrier of the present invention further comprises a drug for treating a disease to be treated, such as a cancer. For example, the drug can be linked with the drug carrier in various ways, so that the drug can be delivered to a target site within the body by administering the drug carrier. Methods for linking the drug carrier with the drug and drugs used to treat specific diseases such as cancers are well known to those skilled in the art. Therefore, the drug carrier of the present invention can further be used to treat diseases such as cancers.

The sixth aspect of the present invention provides a method for treating a disease such as a cancer in a subject by using the drug carrier of the fifth aspect, comprising administering the drug carrier to the subject. In some embodiments, the method further comprises a step of linking the drug carrier with a drug for treating the disease such as cancer, before administering the drug carrier to the subject.

In the above aspects of the present invention, the tumor or cancer is, for example, a CD44 high-expressing tumor including, but not limited to bladder cancer, lung cancer, kidney cancer, gastric cancer, colorectal cancer, liver cancer, breast cancer, melanoma, etc.

The seventh aspect of the present invention provides a method for preparing a targeting GV, comprising the following step: (a) linking a targeting biomaterial with a GV, for example, the targeting biomaterial such as HA is immobilized to a protein shell of the GV by covalent conjugation. In some embodiments, the method further comprises a step (b): linking a biocompatible material with the resulting product from step (a), for example, the biocompatible material such as PEG is chemically conjugated to the product from step (a) by amide formation.

In some embodiments, the step (a) comprises: (a1) mixing the targeting biomaterial such as HA with a phosphate such as sodium phosphate to form a solution; (a2) dissolving GVs in a phosphate such as sodium phosphate solution; (a3) adding 1-(3-dimethylaminopropyl)-3-ethylcarbodiimide (EDC) and N-hydroxysuccinimide (NHS) to the solution from the step (a1) (preferably, pH=7.4); and (a4) adding the solution from step (a2) to the mixture from the step (a3), thereby forming a targeting material-GV (for example, H-GV) conjugate.

In some embodiments, the step (b) comprises: (b1) dissolving the targeting material-GV conjugate (for example, the H-GV conjugate from the above step (a)) in a phosphate buffer (PBS), and mixing the same with EDC in the PBS and NHS in methanol; and (b2) adding the biocompatible material such as monomethoxy PEG-amine to the mixture from the step (b1) to obtain a PH-GV.

The eighth aspect of the present invention provides a lipid GV surface-modified with a lipid molecule. In some embodiments, the lipid molecule(s) is/are distearoylphosphatidylethanolamine-polyethylene glycol (DSPE-PEG), dioleoylphosphatidylcholine (DOPC), PEG, DSPE, polyvinyl alcohol (PVA), glycerol, glyceride, fatty acid, phospholipid or any combination thereof, preferably, the lipid GV is a lipid GV with surface being modified with DSPE-PEG and DOPC. In some embodiments, the lipid GV of the present invention is cylindrical in shape with a particle size of such as about 290-330 nm, for example, about 300-330 nm, about 310-330 nm. The lipid GV has a zeta potential of about −21.3 to −19.3 mV as measured by the dynamic light scattering (DLS) method. The lipid GV of the present invention itself encapsulates certain gases such as air or oxygen within the lipid molecular membrane, but it can be designed to encapsulate/load/carry specific target gases, such as therapeutic target gases, such as oxygen, nitric oxide (NO), hydrogen, etc. Therefore, the lipid GV of the present invention can be used as a carrier for delivering a therapeutic gas to a subject. In this case, the target gas can be encapsulated within the protein shell of the GV by the lipid molecule(s).

Therefore, the ninth aspect of the present invention provides a gas-filled lipid GV comprising the lipid GV of the eighth aspect of the present invention and a gas, for example, the gas can be any type of gases, such as a therapeutic gas. The gas is filled within the protein shell of the GV and encapsulated by the lipid molecule(s). The therapeutic gas described in the present invention includes any gases known in the art that contributes to the treatment, remission, or alleviation of diseases or disease symptoms, or inhibition of progression thereof, including but not limited to oxygen, NO, hydrogen, and the like, preferably oxygen. Therefore, the gas-filled lipid GV of the present invention includes, but is not limited to an oxygen-filled lipid GV, a NO-filled lipid GV, a hydrogen-filled lipid GV and the like, preferably an oxygen-filled lipid GV.

The tenth aspect of the present invention provides a therapeutic agent comprising or consisting of the lipid GV of the eighth aspect of the present invention or the gas-filled lipid GV of the ninth aspect.

The eleventh aspect of the present invention provides a contrast agent or diagnostic agent comprising or consisting of the lipid GV of the eighth aspect of the present invention or the gas-filled lipid GV of the ninth aspect.

The twelfth aspect of the present invention provides a kit comprising the lipid GV of the eighth aspect of the present invention, the gas-filled lipid GV of the ninth aspect, the therapeutic agent of the tenth aspect, or the contrast agent or diagnostic agent of the eleventh aspect, and optionally an instruction for use.

The lipid GV, the gas-filled lipid GV, the contrast agent or diagnostic agent, or the therapeutic agent or the kit of the present invention, all can be used to diagnose or treat diseases, such as hypoxic diseases such as cancers. Therefore, the present invention further provides the use of the lipid GV of the eighth aspect or the gas-filled lipid GV of the ninth aspect such as the oxygen-filled lipid GV or the therapeutic agent of the tenth aspect in preparation of a medicament for treating a disease such as a cancer in a subject. The present invention also provides the use of the lipid GV of the eighth aspect or the gas-filled lipid GV of the ninth aspect such as the oxygen-filled lipid GV or the contrast agent or diagnostic agent of the eleventh aspect in preparation of a diagnostic reagent for diagnosing a disease such as a cancer in a subject. The present invention also provides the use of the lipid GV of the eighth aspect or the gas-filled lipid GV of the ninth aspect such as oxygen-filled lipid GV or the contrast agent of the eleventh aspect in preparation of a diagnostic reagent for tumor imaging in a subject. Where the lipid GV, the gas-filled lipid GV or the contrast agent is used for diagnosing diseases or imaging tumors, it further comprises the application of contrast ultrasound to the subject to determine the presence or absence of a cancer. In some embodiments, the medicament may be used in combination with a second therapeutic agent, such as other drug(s) for cancer treatment or cancer therapy/therapies, so as to treat the cancer. In some embodiments, said cancer therapy/therapies is/are, for example, chemotherapy, radiation therapy, preferably PDT or SDT. Said other drug(s) for cancer treatment may be drug(s) well-known to those skilled in the art for the treatment of various cancers.

The thirteenth aspect of the present invention provides a method for treating a disease such as a cancer in a subject, comprising administering the lipid GV of the eighth aspect or the gas-filled lipid GV of the ninth aspect such as the oxygen-filled lipid GV or the therapeutic agent of the tenth aspect to the subject, or applying the kit of the twelfth aspect to the subject. In some embodiments, the method further comprises administering a second therapeutic agent or therapy, for example, other drug(s) for cancer treatment or cancer therapy/therapies such as chemotherapy, radiation therapy, preferably PDT or SDT to the subject after administration of the lipid GV or the gas-filled lipid GV or the therapeutic agent to the subject, e.g., at 1 min, 5 min, 10 min, 15 min, 30 min, 1 h, 2 h, 3 h, 4 h, 5 h, 6 h, or even 1 day, 2 days, 3 days, 4 days, 5 days, 6 days or 7 days, and any time point between the above numerical values after administration. Said other drug(s) for cancer treatment may be drug(s) well-known to those skilled in the art for the treatment of various cancers. In some embodiments, the method further comprises applying ultrasound to the subject after the administration of the lipid GV or the gas-filled lipid GV or the therapeutic agent to the subject and before the application of the other drug(s) for cancer treatment or cancer therapy/therapies, so as to destroy the lipid GV or oxygen-filled lipid GV and release the gas therein.

The present invention also involves a carrier comprising or consisting of the above-described lipid GV or gas-filled lipid GV such as oxygen-filled lipid GV. The carrier may further comprise a drug for treating a disease such as a cancer (e.g., a drug known in the art for treating a cancer) or a gas such as any therapeutic gas (e.g., a therapeutic gas as defined above). The cancer described in the above aspects of the present invention may be any tumor that can cause hypoxia, such as breast cancer, bladder cancer, lung cancer, kidney cancer, gastric cancer, colorectal cancer, liver cancer, melanoma, and the like.

The fourteenth aspect of the present invention provides a method for preparing a lipid GV, comprising the following steps: (a) dissolving a lipid molecule or a mixture of lipid molecules in chloroform, followed by drying; (b) adding HEPES buffer preferably at pH=7.2 to the product from step (a) under agitation to form a cloudy solution; and (c) adding the product from step (b) to a GV solution to form a lipid GV. In some embodiments, the GV solution is a GV solution in PBS. In some embodiments, the lipid molecule(s) in step (a) is a mixture of DSPE-PEG and DOPC. In some embodiments, the step (b) further comprises sonicating the resulting solution. In some embodiments, the step (c) further comprises adding HEPES buffer to the solution and incubating overnight.

The fifteenth aspect of the present invention provides a method for preparing a gas-filled lipid GV, which is similar to the method of the fourteenth aspect of the present invention, except that the step (c) comprises: filling the GV solution with a gas such as oxygen until saturation prior to adding the product from step (b) to the GV solution.

The sixteenth aspect of the present invention provides a lipid targeting GV surface-modified with a lipid molecule and a targeting biomaterial (and optionally, a biocompatible material). For example, the lipid molecule is one or more selected from the group consisting of DSPE-PEG, DOPC, PEG, DSPE, PVA, glycerol, glyceride, fatty acid, and phospholipid; the targeting biomaterial is one or more selected from the group consisting of HA, RGD peptide, folic acid, galactose, and glucose; and optionally, the biocompatible material is one or more selected from the group consisting of PEG, chitosan, polyurethane, polylactic acid, polyolefin, polysulfone, polycarbonate, and polyacrylonitrile. Preferably, the surface of the lipid targeting GV is modified with DSPE-PEG, DOPC and HA, and more preferably, the lipid targeting GV is surface-modified with DSPE-PEG, DOPC, HA, and PEG. Furthermore, the present invention also provides a gas-filled lipid targeting GV comprising the lipid targeting GV of the present invention and a gas. For example, the gas/gases can be any type of gases, such as a therapeutic gas. The gas/gases is/are filled within the protein shell of the GV and encapsulated by the lipid molecule(s). The therapeutic gas as described in the present invention includes any gas known in the art that contributes to the treatment, remission, or alleviation of diseases or disease symptoms or inhibition of progression thereof, including but not limited to oxygen, NO, hydrogen, and the like, preferably oxygen. The lipid targeting GV or the gas-filled (e.g., oxygen-filled) lipid targeting GV of the present invention can be used as a cancer therapeutic agent, a diagnostic agent, a contrast agent and a drug carrier. Therefore, the present invention also provides the use of the lipid targeting GV or the gas-filled lipid targeting GV of the present invention in the preparation of a medicament for treating a cancer or a diagnostic reagent for diagnosing a cancer. The lipid targeting GV or the gas-filled lipid targeting GV of the present invention can further be used in combination with other cancer therapies or drugs for cancer treatment as defined above in the present invention. The cancer may be a cancer as defined above.

The seventeenth aspect of the present invention provides a method for preparing a lipid targeting GV, which is similar to the method as described in the fourteenth aspect of the present invention, except that the step (c) comprises linking the targeting biomaterial(s) such as HA and GV together prior to adding the product from the step (b) to the GV solution.

The eighteenth aspect of the present invention provides a method for preparing a gas-filled lipid targeting GV, which is similar to the method as described in the fifteenth aspect of the present invention, except that the step (c) comprises linking the targeting biomaterial(s) such as HA and GV together before filling the GV solution with a gas such as oxygen.

The nineteenth aspect of the present invention provides a pharmaceutical composition comprising the targeting GV, the lipid GV, the gas-filled GV, the lipid targeting GV, or the gas-filled lipid targeting GV of any aspect of the present invention.

The present invention also provides a method of treating a cancer in a subject by using the targeting GV, the lipid GV, the gas-filled GV, the lipid targeting GV, the gas-filled lipid targeting GV, or the pharmaceutical composition of any aspect of the present invention. The present invention also provides the use of the targeting GV, the lipid GV, the gas-filled GV, the lipid targeting GV, the gas-filled lipid targeting GV or the pharmaceutical composition of any aspect of the present invention in the preparation of a medicament for treating a cancer in a subject; or the use thereof in combination with a second therapeutic agent or therapy in the preparation of a pharmaceutical combination for treating a cancer in a subject. The targeting GV, the lipid GV, the gas-filled GV, the lipid targeting GV or the gas-filled lipid targeting GV of the present invention can further be used as a diagnostic agent, a contrast agent, a therapeutic agent or a drug carrier. In some embodiments, the method comprises administering the above GV or the pharmaceutical composition to the subject, and then administering a second therapeutic agent or therapy, such as other drug(s) for cancer treatment or cancer therapy/therapies to the subject; alternatively, the method further comprises applying ultrasound to the subject before administering the second therapeutic agent or therapy. In some embodiments, the pharmaceutical combination is implemented as follows: administering the above GV or the pharmaceutical composition to the subject, and then administering a second therapeutic agent or therapy such as other drug(s) for cancer treatment or cancer therapy/therapies to the subject; and alternatively, applying ultrasound to the subject before administering the second therapeutic agent or therapy. Said other drug(s) for cancer treatment or cancer therapy/therapies are those as defined above, and the cancer is a cancer as defined above.

In any aspect of the present invention, the subject may be a human, a non-human primate, a mammal, a rodent, and the like, preferably a human

### BRIEF DESCRIPTION OF DRAWINGS

It should be understood that the drawings described herein are for illustration purposes only. The drawings are not necessarily to scale, with emphasis generally being placed upon illustrating the principles of the present invention. The drawings are not intended to limit the scope of the present invention in any way.

FIG. 1 depicts the morphology, size distribution, and zeta potential of native GVs and lipid GVs. (A) Photographic images of 1 nM GVs. (B) TEM images of GVs, showing their morphology (images are representative). Scale bar represents 100 nm. (C) Size distribution and zeta potential statistics of GVs. (D) Relative size distributions of the two GV groups. (E) GV groups' stability over 6 months observed by measuring their concentration. (F) GV groups' stability over 6 months observed by measuring their size. Data in (E) and (F) represent the mean±standard deviation (SD) on 3 independent experiments.

FIG. 2 depicts the characterization of GVs and PH-GVs. (a) TEM images of GVs and PH-GVs. Due to the wrapping and folding of GVs by PH-HA, we can find the surface of the GVs have been wrapped with a heavy substance. Scale bar represents 200 nm. (b-c) Zeta potentials and Dynamic light scattering analysis of GVs and PH-GVs in PBS at pH 7.4. (d) Ultrasound images of a dropper phantom containing PBS buffer, and GVs and PH-GVs at a concentration ranging from 125 to 1000 pM. Images were acquired at B mode and Contrast mode, as indicated. (e) Quantitative analysis of the images in d. (f) Ultrasound images of a dropper phantom containing GVs and PH-GVs (GVs concentrations of 500 pM) staying for diverse time. (g) Quantitative analysis of the images in f.

FIG. 3 depicts the determination of oxygen-carrying capability of GVs. (A) The oxygen concentration in different concentrations of native GVs after oxygen purge for one minute. Data represent the mean±SD based on 3 independent experiments. *p<0.05 vs. control. (B) The oxygen concentration in different concentrations of lipid GVs after oxygen purge for one minute. Data represent the mean±SD based on 3 independent experiments. *p<0.05 vs. control.

FIG. 4 depicts the determination of oxygen release kinetics of GVs. (A) The oxygen concentration in 5 ml severe hypoxic solution after injection of 1 ml of 1 nM oxygen-filled GVs/oxygen-filled lipid GVs. Data represent the mean±SD based on 3 independent experiments. (B) The oxygen concentration in 5 ml severe hypoxic solution after injection of 1 ml of 1 nM oxygen-filled GVs/oxygen-filled lipid GVs with ultrasound treatment at 5 min for 10 seconds. Data represent the mean±SD based on 3 independent experiments. *p<0.05 vs. control. **p<0.01 vs. control.

FIG. 5 depicts the oxygen-filled GVs mediated oxygen delivery in vitro and in vivo. (A) Represent images of hypoxia condition in cultured cells. 200 μl of oxygen-filled PBS, oxygen-filled lipid GVs, or oxygen-filled GVs (at final concentration of 1 nM) were added into medium and cultured with cells in hypoxic condition for 1 h, respectively. Hypoxia Reagent was used to detect the hypoxia condition in cultured cells, where the fluorescent signal means the occurrence of hypoxia. (B) Quantification of hypoxic staining. Data represent the mean±SD on 3 independent experiments. *p<0.05 vs. control. Scale bar represents 25 μm. (C) 200 μl of 5 nM oxygen-filled GVs, oxygen-filled PBS, or oxygen-filled lipid GVs were injected into the blood vessel of tumor-bearing mice by tail injection, respectively, and oxy-Hb (oxyhemoglobin) and deoxy-Hb (deoxyhemoglobin) levels in the tumor site were monitored by photoacoustic imaging over time. (C) Representative photoacoustic images of tumor oxygen levels at different time points. Red pixels: oxy-Hb; blue pixels: deoxy-Hb. (D) Quantification of tumor oxygen levels. Data represent the mean±SD based on 3 independent experiments. *p<0.05 vs. control.

FIG. 6 depicts the toxicity of GVs both in vitro and in vivo. (A, B) 200 μl of PBS, GVs and lipid GVs (at a final concentration of 1 nM) were added into medium and cultured with cells, respectively, and cell proliferation and LDH toxicity of SCC-7 cells were measured by MTT and LDH assay at different time point, respectively. Data represent the mean±SD based on 3 independent experiments. (C) The score of health condition of mice before and after GVs intravenous injection. Mice were scored on a 30-point scale comprising 10 points each for activity, weight and food intake. Assessment was performed before, immediately after, and 24 h, 48 h and one week after injection (N=3, ±SD). (D) Histological images of major organs with H&E staining collected from mice treated with GVs at day 7. Scale bar represents 100 μm.

FIG. 7 depicts the optical setup for PDT in vitro.

FIG. 8 depicts the cell viability of MCF-7 cells subjected to different treatments after PDT. In vitro cytotoxicity of PDT on MCF-7 cells was determined by CCK-8 assay. Data represent the mean±SEM based on 3 independent experiments. **p<0.01 vs. control.

FIG. 9 depicts the effects of different treatments on cell apoptosis. Upper panel: evaluation of cell apoptosis following PDT was done by flow cytometry through Annexin-V and propidium iodide (PI) double staining. Lower panel: the populations of early apoptotic cells (Annexin-V⁺/PI⁻) and late apoptotic cells (Annexin-V⁺/PI⁺) as a percent of total cells were evaluated. Data represent the mean±SEM based on 3 independent experiments. *p<0.05 vs. control.

FIG. 10 depicts the amount of intracellular ROS in cells after different treatments. Intracellular ROS were stained by DCFH-DA and analyzed by flow cytometry. Left panel: the results of flow cytometry analysis, where the abscissa represents the fluorescence intensity, and the ordinate represents the number of cells; Right panel: the statistical analysis of the results shown in the left panel. Data represent the mean±SEM based on 3 independent experiments. **p<0.01 vs. control.

FIG. 11 depicts the fold increase of SOSG fluorescence intensities of PBS (Control), GV, PpIX and GV+PpIX after ultrasound exposure. Data are represented as mean±SD (n=3). The concentrations of PpIX and GVs are 10 μM and 2 nM, respectively.

FIG. 12 depicts the fluorescence microscopic images of intracellular H₂DCFDA after treatment with PBS (control), GV, PpIX, PpIX+GV with and without ultrasound. Scale bar=20 μm. Excitation light wavelength is 488 nm. Concentrations of PpIX and GVs are 10 μM and 2 nM, respectively.

FIG. 13 depicts (a) cellular uptake of GVs and PH-GVs. Confocal microscopy images of ICG labeled GVs and ICG labeled PH-GV in SCC-7 cells. Scale bar represents 20 μm. (b) Immune escape abilities of GVs and PH-GVs. Cellular uptake in murine RAW 264.7 macrophage cells under a fluorescence microscope. (c, d) Viability assay of SCC-7 cells after treatment with GVs, collapsed GVs and PH-GVs at the concentrations of 0.031-1 nM for 24 h (c) and 48 h (d). The cells treated with PBS were used as control.

FIG. 14 depicts the distribution of GVs and PH-GVs following intravenous administration to tumor-bearing mice. (a) NIR fluorescent imaging of SCC-7 tumor-bearing mice in vivo were taken at different times (in hour) after intravenous injection of ICG labeled GVs and ICG labeled PH-GVs, respectively. Rounds indicate the tumors location. (b) Tumor/muscle (T/M) ratio of SCC-7 tumor-bearing mouse model at diverse time. (c) Fluorescence imaging of major organs and tumor taken from tumor-bearing nude mice ex vivo after 4, 12, 24 and 48 h post-injection of ICG labeled GVs and ICG labeled PH-GVs, respectively. (d) Quantitative analysis for the accumulation in tumor and major organs of ICG labeled GVs and ICG labeled PH-GVs. Error bars represent the SD based on 5 mice per group. *, P<0.05.

FIG. 15 depicts the ultrasound contrast produced by GV at the tumor site. (a) Ultrasound images of tumors in vivo after intravenous injection of GVs and PH-GVs. Green represents the intensity-enhanced region due to the GVs. (b) Quantitative analysis of ultrasound signals from each region of interest in (a). (c) GV collapse and disappearance of ultrasonic signals caused by destructive insonation (650 kPa).

FIG. 16 depicts (a) confocal images of tumor slices collected from mice 12 h after injection of ICG labeled GVs and ICG labeled PH-GVs. The green and red signals are from the fluorescence of ICG and anti-CD31-stained endothelial cells of blood vessels, respectively. (b) Representative H&E sections of the major organs (heart, liver, spleen, lung, and kidney) and tumors after treatment for 30 days. Scale bar represents 100 μm. (c) Weights were measured during the 30-day evaluation period in mice under the different conditions.

FIG. 17 depicts the experimental platform and results of the cavitation reaction, (a) top view of the experimental platform of GV-mediated cavitation reaction; (b) the signal spectrum of the GV and PBS solution.

FIG. 18 depicts a schematic diagram of the application of the surface-modified targeting GV in the diagnosis, imaging, and treatment of tumors according to some embodiments of the present invention.

### DETAILED DESCRIPTION

Previous studies have successfully demonstrated GVs can produce robust ultrasound contrast across a range of frequencies at picomolar concentrations, exhibit harmonic scattering to enable enhanced detection versus background in vivo. However, similar to other nanoparticles, the majority of GVs are up-taken in non-targeted tissues, such as liver, spleen, and lung, following intravenous administration. The pharmacokinetics and biodistribution of nanoparticles are partly determined by their own surface properties. Moreover, a hydrophilic coating is necessary for successful intravenous administration of nanoparticles. The inventors found that chemical surface modification of GVs with biocompatible materials may improve the pharmacokinetic properties and targetability of GVs to increase their tumor accumulation for in vivo molecular imaging. This surface modification is also expected to enhance GVs as good carriers for drug delivery.

In this invention, the surface functionality of GV nanoparticles was modified with biomaterials to achieve improved stability and targetability. The dual-modified GVs were successfully synthesized via the aminocarboxyl reaction. Unexpectedly, the inventors found that chemical modification of GVs surface with biomaterials greatly increased the accumulation of GVs at tumor sites, and thus they may be used as an ultrasound contrast agent or an imaging agent for tumor-specific ultrasound molecular imaging in vivo. The modified GVs can pass through the gaps in the tumor endothelium and enter into the tumor tissue space. These results illustrated that these novel nanoscale ultrasound contrast agents with surface modification exhibited excellent biocompatibility, long blood circulation and remarkable ultrasound contrast in tumor sites. The present invention provided experimental evidence for the future application of GVs in effective extravascular targeted ultrasound molecular imaging and therapy of tumors.

In some embodiments, GVs are surface-modified with a biomaterial with good biocompatibility and targetability, such as polyethylene glycol (PEG)-conjugated hyaluronic acid (HA). PEG on the surface of GVs effectively reduces RES uptake and increases the circulation time in the blood, leading to selective accumulation of the nanoparticles to the tumor site. HA is a biocompatible natural material that is a major extracellular constituent of connective tissues and shows intrinsic targetability to CD44 positive malignant cancer cells. With PEGylated HA, the present invention functionalized GVs (PH-GV) particles, and the technologies of the ultrasound and fluorescence imaging, immunohistochemical sectioning and confocal microscopy were applied to indirectly and directly determine the ability of modified GVs to enter the intracellular space of tumor tissues, demonstrating that the new class nanoscale imaging agents of the present invention can pass through the gaps in the tumor endothelium, and enter the tumor tissue space, and thereby be applied for specific and effective ultrasound molecular imaging and cancer therapy.

As mentioned above, it is believed that tumor oxygenation is helpful for cancer treatment, and oxygen-filled microbubbles can help deliver oxygen to the tissues of interest, thereby improving the therapeutic effects. However, due to limitation of stability and relatively large size, the application of existing oxygen-filled microbubbles is limited. The inventors found that oxyGVs, especially oxyGVs modified with a lipid molecule, can provide enhanced cytotoxicity to tumors in PDT therapy, and had a significant enhancement effect on the therapeutic efficacy of PDT both in vivo and in vitro. Therefore, the novel nanoscale oxyGVs of the present invention could function as an effective oxygen carrier to improve the efficacy of oxygen-consuming PDT. The present invention also investigated the ability of oxyGVs to improve the efficacy of sonodynamic therapy both in vitro and in vivo, and demonstrated that oxyGVs, especially oxyGVs modified with a lipid molecule, could induce enhanced cavitation and excessive production of ROS in cell-free system, and could enhance the production of ROS in vitro, leading to the increased cytotoxicity of SDT. Therefore, oxyGVs can be used as a therapeutic agent to improve the efficacy of SDT.

In the present invention, the term “native GV” refers to a nanoscale gas vesicle isolated from nature, such as algae, without any modification, and “gas vesicle” herein is sometimes referred to as “nanoparticle”. The GVs involved in the present invention include native GVs, lipid GVs, PH-GVs, oxygen-filled GVs, oxygen-filled lipid GVs and other types. “Lipid GVs” refers to GVs surface-modified with a lipid molecule (such as DOPC, DSPE, PEG, etc.). “PH-GVs” and “PH-modified GVs” are used interchangeably, and refer to GVs that are surface-modified with PEG and HA. The terms “oxygen-filled GVs”, “oxygen-GVs” or “oxy-GVs” are used interchangeably, and both refer to GVs filled with oxygen. Similarly, “oxygen-filled lipid GVs”, “oxygen-lipid-GVs”, “oxyGVs modified with a lipid molecule” or “oxy-lipid GVs” are also used interchangeably, which refer to oxygen-filled GVs that are surface modified with lipid molecule(s).

In the present invention, “surface-modified GVs” refer to a class of GVs that are surface-modified with such as lipid molecules, targeting materials, biocompatible materials, etc. and have specific functions such as targetability, hydrophilicity, etc., for example, the PH-GVs and lipid GVs of the present invention are all surface-modified GVs.

### EXAMPLES

### Statistical Analysis

Comparisons among groups were analyzed via independent-samples one-factor ANOVA test using SPASS 17.0 software. All statistical data were obtained using a two-tailed student's t-test and homogeneity of variance tests (p values<0.05 were considered significant).

### Example 1: Preparation and Characterization of GVs and Lipid GVs

### Preparation of GVs

Anabaena flos-aquae (FACHB-I255, Freshwater Algae Culture Collection at the Institute of Hydrobiology, China) were cultured in sterile BO-II Medium at 25° C. under fluorescent lighting with 14 h/10 h light/dark duty cycle. GVs were isolated according to Walsby's method (Buckland and Walsby, 1971). In brief, hypertonic lysis, achieved by quickly adding sucrose solution to a final concentration of 25%, was used to lyse algae to thereby release GVs from the algae. GVs were isolated by centrifugation at 600 g for 3 h. The isolated GVs could form a white creamy layer on top of the solution and can be collected by syringe. GVs were purified three times with phosphate-buffered saline (PBS) and stored in PBS at 4° C.

GV concentration was estimated using a literature-based formula (450 nM per OD500) (Walsby, 1994), where OD500 is the optical density at a 500 nm wavelength measured with a UV-visible spectrophotometer (2100 Pro, GE Healthcare, Piscataway, N.J., USA). Volume fraction was estimated using approximated gas volumes of 8.4 μL/mg and molar weight of 107 MDa as described previously (Walsby and Armstrong, 1979). GV morphology was imaged using a transmission electron microscopy (TEM) (JEOL 2100 F; JEOL, Tokyo, Japan) operating at 200 kV. GV samples in deionized water (0.5 nM) were deposited on a carbon-coated grid and dried at room temperature overnight. Hydrodynamic size was obtained using the dynamic light scattering (DLS) method.

### Preparation of Lipid GVs

A total amount of 1 μmol of lipid mix containing DOPC, DSPE-PEG (2000) (4:1), was dissolved in around 100 μL chloroform in a 25 ml round-bottom flask. The solvent was then evaporated and the samples were dried in a vacuum rotary evaporator. 1 mL of 20 mM HEPES buffer (pH=7.2) was then added to the lipid dry layer, forming a cloudy solution after vigorous agitation. The mixture was then sonicated (20 W, 15 s pulses for 20 min, each pulse separated by a 30 s dead time) for 3-5 min until the solution became clear. The resulting liposome solution was stored at 4° C. until further use. A volume of 0.5 mL of the prepared liposome solution was added to the GV solution. The volume was increased to 1 mL with the same 20 mM HEPES buffer. The mixed solution was incubated overnight on a rocker. After that, GVs were washed three times through centrifugation (2.4 krpm, for 10 min) and resuspended in Milli-Q water. Finally, lipid GVs were resuspended in PBS and ready to use.

Size distribution and zeta potential determination: Size distribution and zeta potentials of lipid GVs were determined by laser light scattering using a 90 Plus instrument (Brookhaven, Holtsville, N.Y., USA) at a fixed angle of 90° and a temperature of 25° C.

Morphological analysis: The size and morphology of lipid GVs were determined by Transmission Electron Microscopy (TEM) with the operating voltage of 200 kV. Samples of lipid GVs (OD 0.1) were deposited on a carbon-coated formvar grid and stained with 2% uranyl acetate.

Particle stability: To measure the stability of lipid GVs formulations, single particle size and concentration of liposome encapsulated GV formulations were determined at 1, 3, and 7 days after fabrication, respectively.

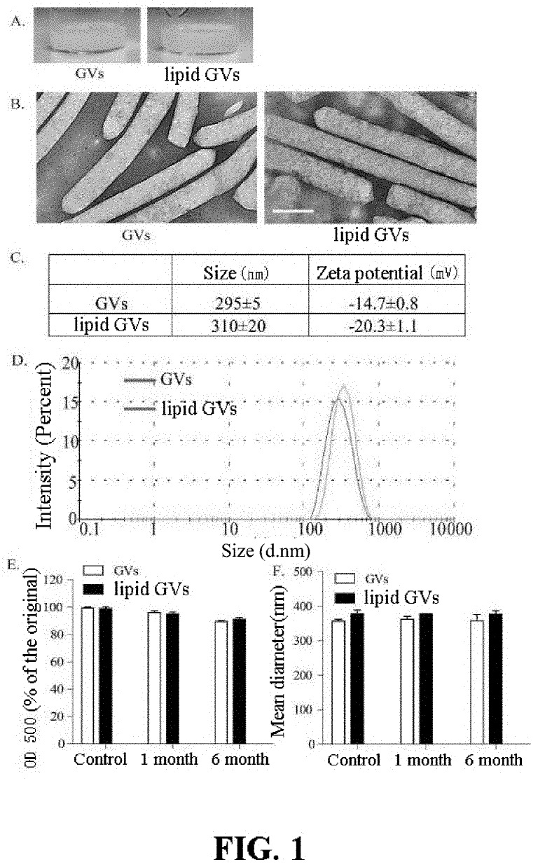

GVs used in this study were produced by culturing the algae Anabaena flos-aquae, from which GVs were isolated by centrifugation. Since the shells of GVs are permeable to gas molecules, this might affect its oxygen delivery efficiency. Therefore, we prepared two kinds of GVs: native GVs and GVs surface-modified with lipid(s) to reduce gas exchange (lipid GVs) (C. Zhang et al., Colloids Surf. B Biointerfaces 160 (2017) 345-354; C. Zhang et al., ACS Appl. Mater. Interfaces 10 (1) (2018) 1132-1146). When presented in solution (FIG. 1A) or in the form of individual vesicles observed by TEM (FIG. 1B), there was no major visible difference between native GVs and lipid GVs. Next, lipid GVs were characterized by particle size distribution and zeta potential. We found that these nanobubbles had an average diameter of about 300-330 nm and a uniform distribution (FIGS. 1C-1D). The lipid GVs had an average particle size of about 290-330 nm (FIG. 1C) or about 310-330 nm (measured by DLS, FIG. 1D), which was about 10 nm larger than native GVs. The lipid GVs had a zeta potential of about −21.3 to −19.3 mV, with a lower negative charge than that of GVs (FIG. 1C). We further evaluated the stability of GVs under cold storage (4° C.), and observed the concentrations (determined by OD500) and sizes of the two groups (the GVs group and the lipid GVs group) from zero to six months and found no significant difference to either group (FIGS. 1E-1F). Therefore, we were able to produce nanoscale negatively-charged GVs that were stable in solution through long-term storage.

### Example 2: Preparation and Characterization of HA-GVs and PH-GVs

### Preparation of HA-GVs (H-GVs)

HA were immobilized to GVs' protein shell by covalent conjugate. For the HA-GV synthesis, firstly, EDC (3.37 mM) and NHS (2 mM) were added to HA solution (10 mg) in 0.1 M sodium phosphate (pH=7.4). The solution mixture was stirred at ice-bath for 2 h and was added dropwise with 6 mL of GVs dissolved in sodium phosphate (pH=7.4). The reaction mixture was stirred at 4° C. for another 24 h. The resulting mixture was added into the ultrafiltration tube (50 mL) and centrifuged at 1.8 rpm for 5 min to remove free EDC, NHS and HA. The resultant nanoparticles were stored in PBS buffer at 4° C.

### Preparation of PEGylated HA-GVs (PH-GVs)

PEG was chemically conjugated to the H-GV conjugate, through amide formation by varying the feed ratio of PEG to HA in the presence of EDC and NHS. The H-GV conjugate (120 mg) was dissolved in phosphate buffer (PBS, pH 7.2), which was mixed with EDC (3.37 mg) in PBS and NHS (2 mg) in methanol. After monomethoxy PEG-amine (73.5 mg) was slowly added, the mixture was stirred at ice-bath for 24 h. Then the resulting solution was centrifugated to remove the excess amount of methanol and PEG and washed with PBS for 4 times.

For the cellular experiments and animal imaging tests, the GVs were labelled with an NIR dye (ICG) at first step before adding HA and PEG. Briefly, EDC (3.37 mg) and NHS (2 mg) were added to ICG solution (1 mM) in 0.1 M sodium phosphate (pH=7.4). After 30 min reaction at room temperature, the solution was added to pure GV solution (molar ratio: ICG/GV=1000/1). Then the mixture was shaken for 4 h at room temperature and followed by purification by centrifugation for 4 times. The resulting mixture was added into the ultrafiltration tube (50 mL) and centrifuged at 1.8 rpm for 5 min to remove free ICG. The resultant nanoparticles were stored in PBS buffer.

The particle size and size distribution of PH-GV NPs were measured by dynamic light scattering DLS (Varian, Palo Alto, USA). Zeta potential measurements were performed at 25° C. on a Malvern Zeta Size-Nano Z instrument. The nanostructure and size of PH-GVs were observed by Transmission electron microscopy (TEM) (Bruker, Germany). UV-vis absorbance spectra of GVs and PH-GVs were observed by Multiskan Go microplate reader (Thermo Fisher Scientific, Massachusetts, USA). Fluorescent signals of ICG and ICG labelled PH-GVs were measured using a fluorescence spectrophotometer (Varian, Palo Alto, USA).

The Ultrasound signals test of GVs and PH-GVs: PH-GVs and GVs containing the same amount of GVs were subjected to US imaging. PH-GVs and GVs were put into dropper (5 mL) before imaging and all the droppers were immersed at the same depth in the deionized water. Ultrasound B-mode and Contrast mode images of the GV solution were acquired using a high-frequency ultrasound system with a transducer of LZ250 D. The center frequency and output energy level were set to 18 MHz and 4%, respectively.

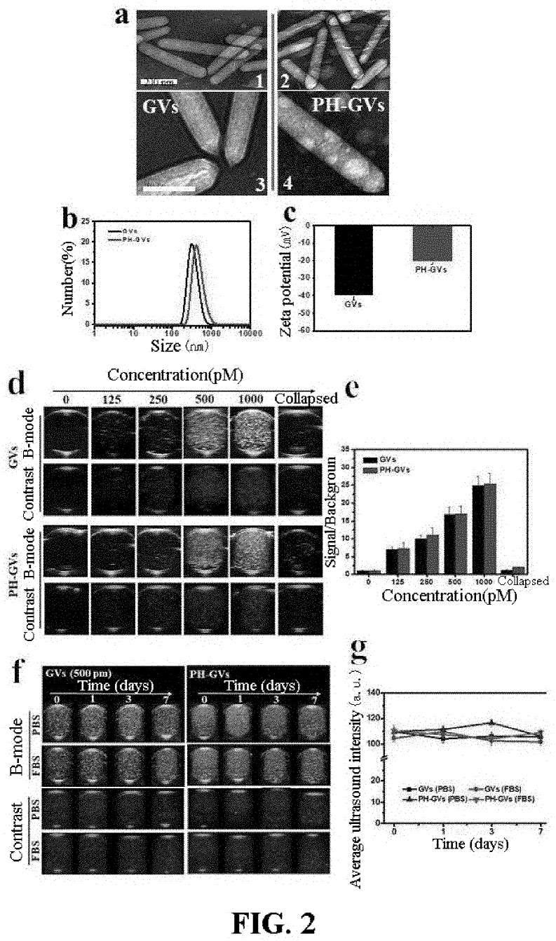

To achieve optimal stability and targetability, the surface functionality of GV nanoparticles was modified. The dual-modified GVs were successfully synthesized via the Aminocarboxyl reaction. Firstly, GVs were surface-modified with hyaluronic acid (HA), one of polysaccharides consisting of N-acetyl glucosamine and glucuronic acid repeating units, to increase the biocompatibility and targetability of GVs. Then PEG-5000 was readily attached to the backbone of the HA conjugate through amide formation in the presence of EDC and NHS, which can make the HA-GVs stealth from the host's immune system by hindering non-specific interaction with plasma protein, can prolong its circulatory time by reducing reticuloendothelial system (RES) clearance and can enhance the stability of colloidal dispersions via steric repulsion of hydrophilic PEG chains. The successful synthesis of PH-GVs was confirmed by transmission electron microscopy (TEM). As shown in FIG. 2a, TEM revealed a cylindrical morphology of naked GVs with a mean particle size of 400 nm. Due to the wrapping and folding of GVs by PH-HA, we found the surface of the GVs had been wrapped with a heavy substance (FIG. 2a). Dynamic light scattering (DLS) reported that the particle size was about 320-380 nm for GVs, and about 375-425 nm for PH-GVs; the zeta potential was in the range of −40 to −50 mV for GVs, and about −27±4 mV for PH-GVs (FIG. 2b).

In addition, with the coating of PEG, zeta potential of the pegylated HA-GVs decreased due to the shielding function of PEG layer. As shown in FIG. 2a-b, PH-GV had a cylindrical morphology with radii about 400 nm, and all nanoparticles were cylindrical in shape and showed a relatively uniform size distribution. The suitable particle size and negative zeta potential of PH-GV nanoparticles (NPs) ensured good tumor-targeting cargo delivery via EPR effect, while reducing RES clearance.

In view of the strong ultrasound property of GVs and PH-GVs, GVs from Anabaena flos-aquae (Ana) were purified through tonic cell lysis and centrifugation assisted flotation, which were imaged in a Vevo 2100 imaging system (FUJIFILM VisualSonics, Toronto, Ontario, Canada; B mode) operating at 21 MHz at different concentrations (for imaging details, see Example below). GVs produced robust contrast relative to buffer controls at concentrations ranging from 250 pM to 1 nM (FIGS. 2c-2d), and gas volume fractions of about 0.01% to 0.1%. Contrast was strongest at the highest concentration, with Ana GVs producing 23.4±2.5 greater scattering than buffer controls (FIG. 2e). Moreover, the ultrasonic performance of GVs was not affected after PEG and HA modification. Before the next experiment, the biological stability of PH-GVs was identified in PBS and fetal bovine serum (FBS). GVs and PH-GVs still had good ultrasound imaging capabilities after being placed for more than one week (FIG. 20, and showed good stability (FIG. 2g).

### Example 3: Determination of Oxygen-Carrying Properties and Oxygen Release Kinetics of GVs and Lipid GVs

The oxygen concentration in the solution was monitored using an oximeter (Portamess 913 OXY; Knick, Germany) and the data were recorded as mg/l. Different concentration of GVs and lipid GVs were purged with oxygen/nitrogen and the oxygen concentration of different solutions was determined, respectively.

Oxygen release without ultrasound: The oxygen concentration of PBS, sealed into vials, was reduced to 0.8 mg/l (severe hypoxia) with an N₂ purge, in order to mimic hypoxic conditions. PBS, GVs and lipid GVs were saturated with an oxygen purge. The concentration of oxygen released by diffusion from GVs/lipid GVs into the hypoxic solution was monitored over time. Before each experiment, the oxymeter was calibrated. All the experiments were performed in triplicate.

Oxygen release with ultrasound: To investigate the effect of ultrasound (US) on oxygen release of GVs/lipid GVs, a US probe with an oscillation frequency of 1 MHz and an average acoustic pressure distribution value of 2.4±0.2 MPa (nominal frequency: 50 Hz; and nominal power: 30 W) was used. The time for ultrasound treatment was 10 s. The change of oxygen concentration in the solution was monitored after ultrasound treatment. All the experiments were performed in triplicate.

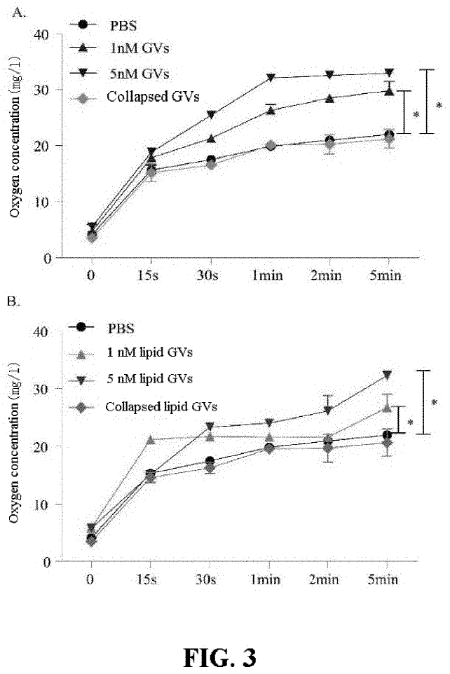

The relative oxygen-carrying properties of the two GV groups (GV and lipid GV group) were compared. As these solutions were purged with oxygen, the oxygen concentrations of both GV groups increased continuously and finally became stable. The oxygen carrying capability of GVs was found to be much higher than PBS and that of broken GVs, and the overall capacity was dependent upon GV concentration (FIG. 3A). Lipid GVs also showed similar patterns of greater oxygen carrying capacity compared to PBS or broken GVs, and a dependence upon concentration (FIG. 3B).

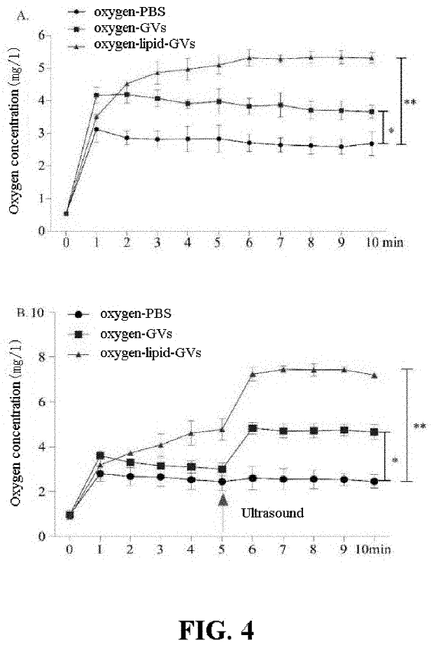

The oxygen release kinetics of different GVs was detected. We found that both oxygen-filled GVs and oxygen-filled lipid GVs could increase the oxygen concentration in severely hypoxic solutions significantly as compared to PBS (FIG. 4A). Compared to oxygen-filled GVs, the release of oxygen by oxygen-filled lipid GVs into solution was slower. We believed that this was due to the surface modification of GVs leading to slower oxygen release kinetics. In addition, the total released oxygen for oxygen-filled lipid GVs was greater (area under the curve), which can be explained by the presence of lipid(s) on the surface of GVs as lipid(s) reduced the release of oxygen by GVs. This property might also increased oxygen delivery efficiency during long transportation times in vivo. The lipid GVs were therefore comparable to the GVs in their oxygen-carrying capacities, but showed slower release patterns, and were able to increase the oxygen concentration to a greater degree. We also added a 10 second sonication step using ultrasound to trigger greater oxygen release. The ultrasound had no effect on oxygen release by PBS, but greatly increased that by the GV groups (FIG. 4B).

### Example 4: Determination of the Capacity of Oxygen-Filled GVs and Oxygen-Filled Lipid GVs to Deliver Oxygen

Cells were cultured and passaged in a hypoxic chamber (1% oxygen, 5% CO₂). The hypoxic conditions in the media before/after the addition of oxygen-filled lipid GVs were monitored using Image-iT™ Red Hypoxia Reagent purchased from Thermo Fisher.

In vivo experiments: All procedures using laboratory animals were approved by the Department of Health, The Government of the Hong Kong Special Administrative Region and the Hong Kong Polytechnic University Animal Subjects Ethics Sub-committee. 18-20 g female Balb/c mice were supplied by the Animal Resource Centre of Hong Kong Polytechnic University. The mice were acclimated to the room for one week after arrival and were maintained on a normal 12 h light-dark cycle. The mice were housed in conventional cages (6 animals per cage) with free access to standard pellet diet and water in specific pathogen-free condition at the temperature of 24±2° C. and 60-70% relative humidity. Standard wood chips for mice were used as bedding material. After 1 week's acclimation, 0.1 ml SCC-7 cell line (1*10⁷ cells/ml) were resuspended in 100 μL matrigel and implanted into the rear dorsal of Balb/c mice by subcutaneous injection. Tumor formation occurred approximately two weeks after cell implantation and the tumor showed a significant degree of hypoxia at the stage where the volume reached around 200 mm³.

Measurement of oxy/deoxy-Hb levels using photoacoustic imaging: After oxygen-filled lipid GVs were injected into mice, the oxy- and deoxy-Hb levels in subcutaneous tumors were monitored through Vevo LAZR photoacoustic imager (Fujifilm Visualsonics, Amsterdam, the Netherlands) with a hybrid US transducer (central frequency=21 MHz; spatial resolution=75 μm). pO₂ levels were recorded and stored for later comparison between groups.

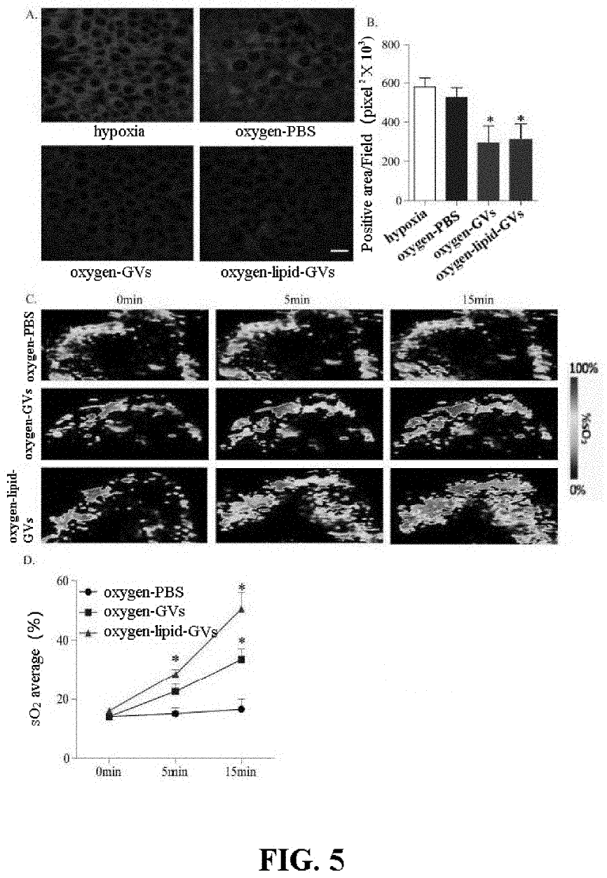

The abilities of each GV group (oxygen-filled GVs and oxygen-filled lipid GVs) to modify the hypoxic conditions of cells in vitro and in tumor masses in vivo were further evaluated. Human breast cancer MCF-7 cells were grown for 24 h under hypoxic conditions, and their levels of hypoxia were monitored using Image-iT Red Hypoxia Reagent. Compared to the untreated control and PBS, the addition of oxygen-filled GVs and of oxygen-filled lipid GVs significantly reduced the observed levels of hypoxia (FIGS. 5A-5B).

We also performed an in vivo proof-of-concept study to determine the ability of oxygen-filled GVs injected into the tail-vein to elevate hypoxic subcutaneous tumor oxygenation levels in nude mice. Tumor oxygenation was monitored by visualizing the levels of oxy-Hemoglobin (oxy-Hb) and deoxy-Hemoglobin (deoxy-Hb) through photoacoustic imaging before and after the treatment (0, 5, and 15 min). Injection of both oxygen-filled GVs group and oxygen-filled lipid GVs group resulted in elevated oxy-Hb levels in the tumors, but not with the PBS control (FIG. 5C). Furthermore, addition of oxygen-filled GVs raised oxygen saturation (sO₂) in the tumor by 20% compared to the control, but addition of oxygen-filled lipid GVs raised sO₂ by 50% (FIG. 5D).

The surface modification of GVs with lipids thus showed significantly better results in our preliminary experiments at increasing oxygen levels in in vivo settings, thus demonstrating their increased stability and oxygen-delivery abilities.

### Example 5: In Vitro and In Vivo Toxicity Detection

Cell viability and LDH assay: Human breast cancer cell lines (MCF-7) were obtained from the cell bank of the Chinese Academy of Science, Shanghai, China. MCF-7 cells were cultured in high-glucose (4.5 g/l) Dulbecco's modified Eagle's medium with L-glutamine following standard cell culture instructions. All media were supplemented with 10% (vol/vol) fetal bovine serum, penicillin (100 U/mL) and streptomycin (100 μg/ml). Cells were grown at 37° C. in a 5% CO₂ and 95% air atmosphere until 70%-80% confluence before trypsinization and harvesting for both cell culture and in vivo studies. After that, different doses of lipid GVs (1 nM) were added to the cell culture media at different time points: 24 h, 48 h, and 72 h. After that, LDH assay was performed using Pierce LDH Cytotoxicity Assay Kit (Life Invitrogen) according to the manufacturer's instructions. Cell viability and apoptosis were determined by MTT assay and apoptosis assay according to the manufacturer's instructions.

In vivo toxicity determination: Post-mortem exams were performed in lipid GVs treated mice and tissue samples (livers, lungs, and kidneys) were collected for histology. The liver, lung, and kidney samples collected from the mouse bioassays were fixed in 10% buffered formalin, processed through conventional histological techniques, and stained with hematoxylin and eosin. Microscopy was performed using an optical microscope (Olympus BX51) equipped with a camera (Olympus Q-Color-5), and the images were recorded in a computer using the Image Pro-Express software.

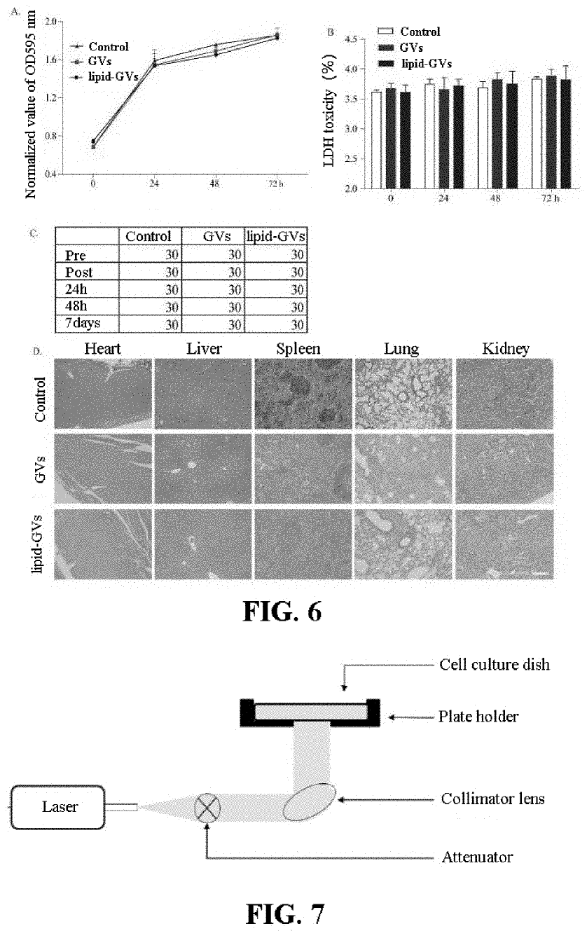

To test the toxicity and biosafety of the two GV groups (the GV and lipid GV groups), we used the LDH and MTT assays in vitro and found that neither GV group triggered significant cytotoxicity. GVs or lipid GVs (final concentration=1 nM) were added to the culture medium and allowed to incubate for 24, 48 or 72 h. No significant elevation in LDH or MTT levels was observed in either GV condition, compared to the control (FIGS. 6A-B). We next tested the biosafety of the GV groups in vivo by observing three basic measures of mouse health (activity, weight and food intake) before GV administration, and 24, 48 and 72 h after administration of GVs. Scoring the mice on a 30-point scale, we observed no decrease in these indicators over the time period (FIG. 6C). We also assayed the mice's major organs (hearts, livers, spleens, lungs and kidneys) one week after GV administration using hematoxylin and eosin (H&E) staining. Tissue slices from both GV groups presented no significant pathological abnormalities or lesions compared to the control group (FIG. 6D). We thus determined that the GVs and lipid GVs were not cytotoxic to cells and did not cause any significant damage to the mice in which they were tested.

### Example 6: Effect of the Combination of oxyGV and PDT on Cytotoxicity and Apoptosis

Photodynamic therapy setup: Cells were exposed to laser with the power of 100 mW/cm² for 5 min. The optical setup for PDT treatment was shown in FIG. 7. The light source with a wavelength of 396 nm that was generated by an optical fiber was collimated to an aperture and irradiated to the 35 mm cell culture dish. The position of the cell culture plate was manually controlled by a dual-axis electric linear platform. After the treatment, cells were cultured in fresh medium for 4 h and then prepared for different analyses.

Cell culture: Human breast cancer cell line (MCF-7) was obtained from the Shanghai Cell Bank. The MCF-7 cells were grown in a high-glucose Dulbecco's modified Eagle's medium (DMEM, 4.5 g/L D-glucose) supplemented with 100 U/ml penicillin-streptomycin containing 10% fetal bovine serum (FBS) at 37° C., in 5% CO₂ humidified atmosphere.

Cell viability test: In the pilot study, MCF-7 cells were divided randomly into eight groups: (1) control, (2) laser alone, (3) PpIX alone, (4) PpIX plus laser (PDT), (5) PpIX+GVs+laser (PDT+GVs), (6) PpIX+oxyGVs+laser (PDT+oxy-GVs), (7) GVs+laser, and (8) oxyGVs+laser. For the PpIX treatment, cells were incubated with 10 μM PpIX for a 4 h drug-loading time in DMEM medium supplemented with 10% FBS. Cell viability at the different time points following PDT was determined using a Cell Counting Kit-8 (Sigma-Aldrich) according to the manufacturer's instructions. Briefly, cells were plated at a density of 5000 cells per well in a 96-well plate and incubated in 100 μL culture medium for 24 h. Cytotoxicity was determined by adding 10 μl CCK-8 reagent per well for 1 h at 37° C. in 5% CO₂. The absorbance of the treated samples against a blank control was measured at 450 nm as the detection wavelength. The viability of treated cells was determined by comparing to the untreated ones in the control group.

Cell apoptosis test: Cells were seeded at a density of 5×10⁵ cells in 6-cm dishes and incubated for 24 h. Alexa Fluor 488 Annexin V/Dead Cell Apoptosis Kit (Thermo Fisher Scientific) was used to measure cell apoptosis according to the manufacturer's instructions. Cells were collected and incubated with 5 μL of the annexin V conjugate and 1 μL of the PI working solution at room temperature for 15 min. The cells were analyzed by FACS Calibur flow cytometer and BD Accuri C6 Software (Becton-Dickinson, USA).

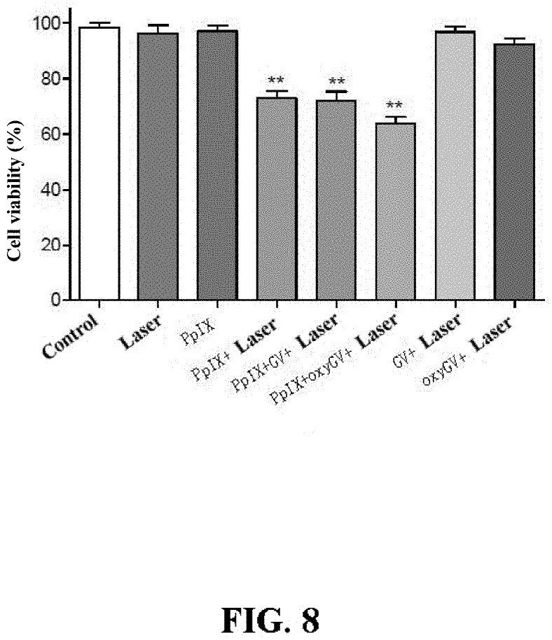

FIG. 8 depicts effects of different treatments on cell viability. In vitro cytotoxicity of different treatments on MCF-7 cells was determined by CCK-8 assay. Data represented the mean±SEM based on 3 independent experiments. **p<0.01 vs. control.

To determine whether oxyGVs could increase the efficacy of PDT, the cytotoxicity of ALA-PDT on MCF-7 cell lines were determined using a CCK-8 assay at 4 h following PDT treatment as shown in FIG. 8. We found that ALA-PDT (PpIX+laser) decreased cell viability of MCF-7 cells markedly to 75% at 4 h following PDT (FIG. 8, p<0.05). Cell viability of MCF-7 cells under ALA-PDT treatment decreased to 62% with the addition of oxyGVs while native GVs (GVs without oxygen) had little effects on ALA-PDT induced cell death. At the same time, other groups, including PpIX alone, laser alone, GVs plus laser, and oxyGVs plus laser groups, showed no effects on cell viability. Due to the negligible effects of these groups, cells were divided into four groups (1) control, (2) PpIX plus laser (PDT), (3) PpIX+GVs+laser (PDT+GVs), and (4) PpIX+oxyGVs+laser (PDT+oxy-GVs) in the following experiment.

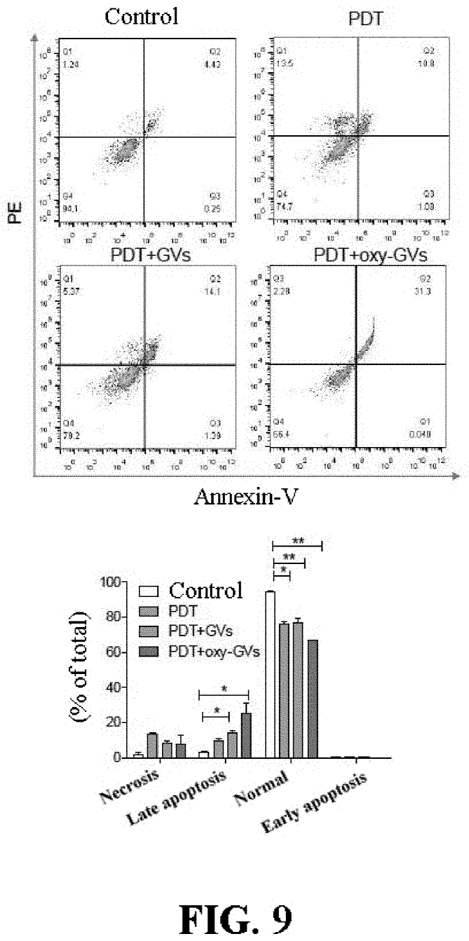

We next investigated effects of oxyGVs on ALA-PDT induced cell apoptosis by flow cytometry as shown in FIG. 9. In each of the upper panels of FIG. 9, cells in the lower-left quadrant represented viable cells, both the lower-right and upper-right quadrant showed the apoptotic cells, and the necrotic cells appeared in the upper-left quadrant. As shown in the lower panels of FIG. 9, there were 94.1%, 74.7% and 79.2% viable cells in control, PDT and PDT+GVs groups, respectively. However, only 66.4% viable cells were detected in PDT+oxyGVs group. This means that the addition of oxyGVs results in a significant higher death and apoptosis rate during PDT.

### Example 7: Effect of OxyGVs on ROS Production During PDT

Intracellular ROS test: Intracellular ROS production was measured using DCFH-DA (Sigma-Aldrich). Briefly, 10 μM DCFH-DA diluted with PBS were added to MCF-7 cells at 37° C. for 20 min. Cells were then washed with PBS three times. Labeled cells were trypsinized and analyzed by flow cytometry.

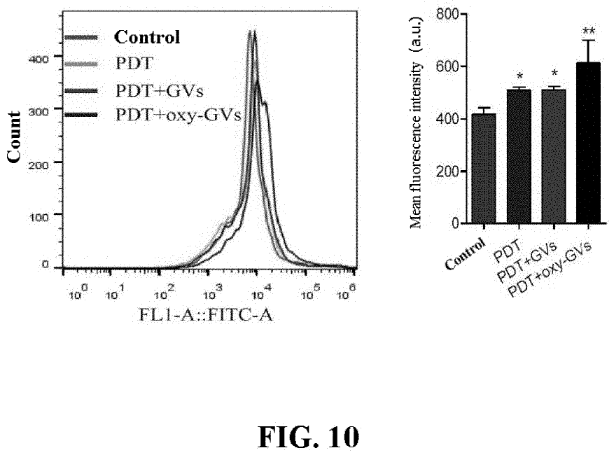

Excessive production of ROS was believed to be responsible for the cytotoxicity to tumor cells during PDT. Thus, we further investigated whether intracellular ROS increased following PDT using flow cytometry and results were shown in FIG. 10. As expected, excessive ROS production was detected in all three PDT treatment groups, while total intracellular ROS were significantly increased with the addition of oxyGVs compared with other groups.

### Example 8: Singlet Oxygen Generation in Cell Free System

SDT setup: 1 MHz planar ultrasound transducer with diameter of 5 cm (Olympus, Tokyo, Japan) was used for sonification in this study. Function generator (AFG 3251, Tektronix Company, Oregon, USA) and power amplifier (Model 500 A250 CAR; Souderton, Pa., USA) were used to generate ultrasonic pulses. Cells were exposed to ultrasound 10 cm away from transducer coupled by degassed deionized water at 25° C. Acoustic intensity and field were characterized by hydrophone (HGL-200; Onda, Sunnyvale, Calif., USA). The spatial peak temporal average intensity was measured to be 8 W/cm²; the duty cycle was 20% with fixed pulse repetition frequency (PRF) of 1000 Hz. The overall sonification duration was 5 min. The temperature increase in cell-culture experiment solution was controlled within 2.5° C. After the ultrasound treatment, cells were cultured in fresh medium for 4 h and then prepared for different analyses.

Generation of singlet oxygen: SOSG (10 μM) was used to detect singlet oxygen generation. The solution was exposed to ultrasound and protected from light during the exposure time. The fluorescence intensity of SOSG was measured by microplate reader at an excitation wavelength of 488 nm and an emission wavelength of 530 nm.

To investigate whether GVs could enhance ROS production during SDT, we tested singlet oxygen production in cell free system under ultrasound exposure using fluorescent probe of SOSG as results were shown in FIG. 11. For singlet oxygen detection, we noticed that the percentages of SOSG fluorescence intensity increased significantly before and after ultrasound treatment in PpIX alone (10 μM), GVs alone (2 nM), and PpIX plus GVs groups. Notably, the addition of GVs with PpIX lead to more than twice as effective at producing singlet oxygen compared to PpIX alone. These results inferred the synergic effect between GVs and PpIX under ultrasonic exposure to enhance the singlet oxygen production.

### Example 9: Effect of GVs on ROS Production During SDT

Refer to the above Example 7 for the test method.

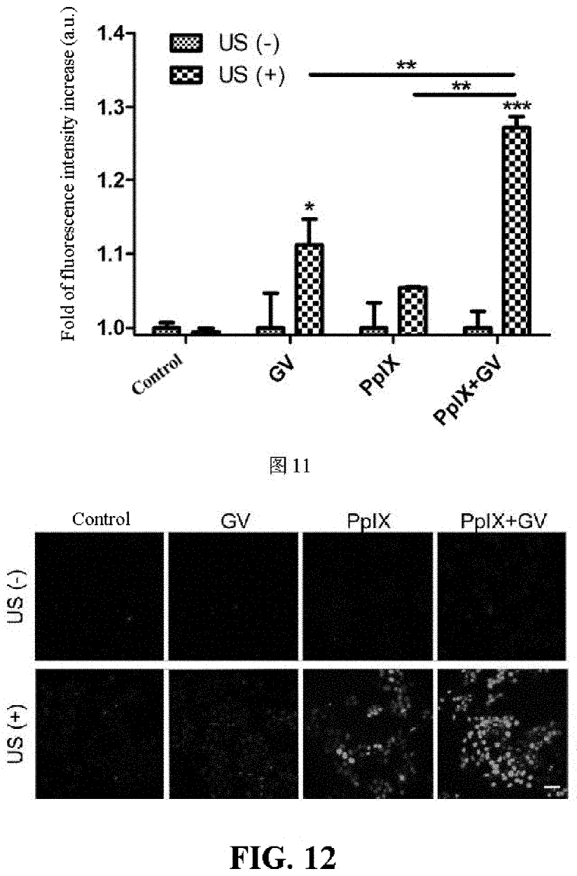

Excessive production of ROS was believed to be responsible for the cytotoxicity to tumor cells during SDT. Thus, we further investigated whether intracellular ROS increased following SDT using fluorescent imaging as shown in FIG. 12. As expected, excessive ROS production was detected in all three SDT treatment groups, while total intracellular ROS were significantly increased with the addition of GVs compared with other groups.

### Example 10: Cavitation Characterization

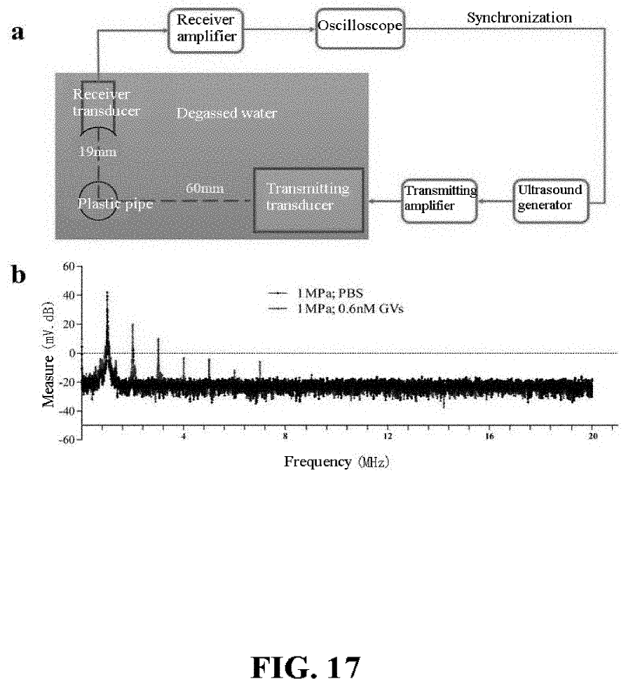

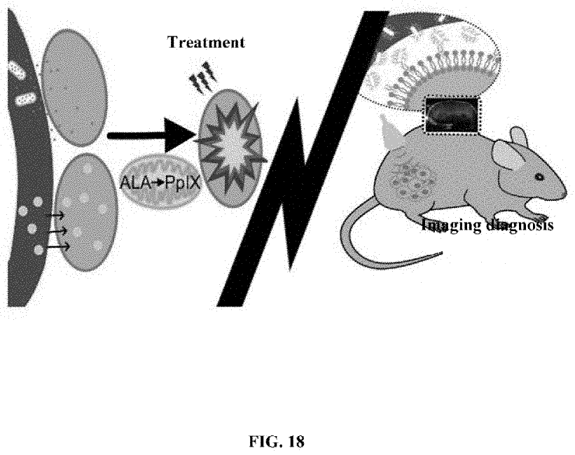

Cavitation intensity was characterized by passive cavitation detection (see FIG. 17). A planar single element ultrasound transducer (center frequency=5 MHz, bandwidth=4.8-5.2 MHz), as a receiver, was placed perpendicular to the treatment ultrasound transducer and confocally aligned with it. It was synchronized with 1 MHz treatment transducer. During each experiment, the FUS transducer sent out ultrasound pulses to the medium with or without GVs (concentration=2 nM). The ultrasound parameter was kept the same as what used in cell-culture and ROS characterization experiments. GVs were filled in a 5 mm agar-gel cubic camber placed at the confocal region of FUS transducer and receiver transducer. RF data of the receiver transducer was acquired by oscilloscope (LeCroy 715 Zi; LeCroy, Chestnut Ridge, N.Y., USA) during the sonication of each FUS pulse. The acquired RF data was transferred to frequency spectrum and inertial cavitation intensity was calculated by integration within broadband signal (4.8 MHz-5.2 MHz).

### Example 11: Targetability and Immune Escape of GVs and PH-GVs

Cell culture and cell internalization of GVs and PH-GVs: Human squamous cell carcinoma cell line (SCC7 cells) and NIH-3T3 cells were cultured in an 8-well chamber in Dulbecco's Modified Eagle Medium (DMEM)/high glucose medium supplemented with 10% FBS and 1% antibiotic solution at 37° C. and 5% CO₂. The next day, both cells were washed by cold PBS and incubated with IGV at 37° C. for 4 h with 5% CO₂ atmosphere. After incubation, all cells were washed with cold PBS thoroughly. Cells were finally fixed in cold ethanol for 15 min at −20° C., and then mounted with mounting medium containing DAPI for 10 min in the dark. Cell internalization of ICG labelled PH-GVs and ICG labelled GVs were observed by a confocal microscope (Olympus, USA) and the excitation and emission wavelengths were set at 780 nm and 800 nm for ICG, respectively.

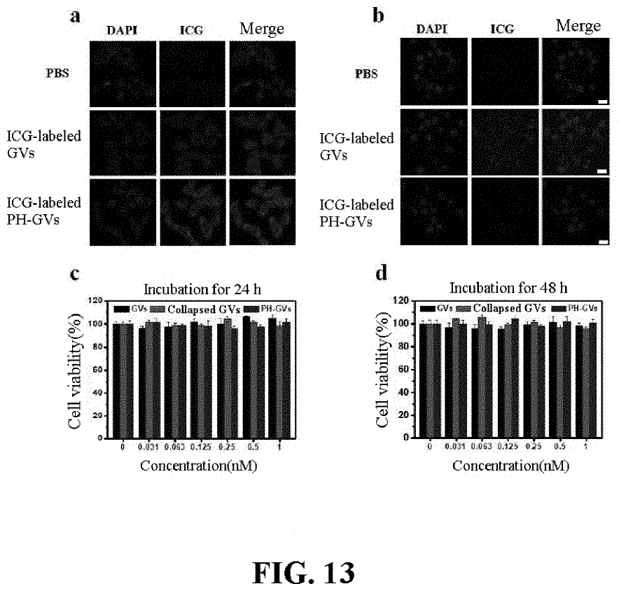

To investigate the targeting efficiency of PH-GVs to CD44 high-expression tumor cells, they were labeled with the NIR fluorophore, ICG, followed by incubation with SCC7 cancer cell lines and NIH3T3 cells. The cancer cells, used in this study, had been demonstrated to over-express CD44 on their surfaces, compared to low expressed cell lines (NIH3T3). Cells, fixed at pre-determined timepoints, were then examined using the Confocal Laser Scanning Microscope (CLSM). FIG. 13a demonstrated the CLSM images of SCC7 cells after 6 h incubation with PBS solution, ICG labeled GVs and ICG labeled PH-GV nanoparticles. As shown from the CLSM images, ICG intracellular distribution of PH-GV nanoparticles was quite different from GV solution. After 6 h incubation with ICG labeled PH-GV solution, the significant fluorescence of ICG distributed in the whole cytoplasm, while SCC7 cells treated with ICG labeled GVs were not observed with strong red fluorescence in the cytoplasm.

The cellular uptake of PH-GV and GV nanoparticles were further evaluated in murine RAW 264.7 macrophage cells by fluorescence microscope (FIG. 13b). PBS was used as a control. The captured images showed that free GVs were extensively internalized in macrophage cells with strong red fluorescence. However, in PH-GV nanoparticles group, there was a decreased red fluorescence signal from the macrophage cells. The FACS analysis showed that the internalization of PH-GV nanoparticles in macrophages was reduced about 40% compared with the free GVs group. Accordingly, the PEG surface decoration of PH-GV nanoparticles could obviously reduce the internalization in macrophages.

### Example 12: Biosafety of GVs and PH-GVs

Animals were sacrificed when the volume of their tumors reached 2000 mm³ according to the protocol of the animal study. To contrast the treatment effect, a part of tumors in all groups were collected for hematoxylin-eosin (H&E) staining after 2 days post injection.

To evaluate the biosafety of GVs, the major organs (hearts, livers, spleens, lungs, kidneys) were collected and examined by H&E staining post 14 days of treatment.

Effects of free GVs and PH-GV nanoparticles on cytotoxicity and apoptosis were investigated at diverse concentration (GVs, 0-1 nM) in SCC7 cancer cells. As shown in FIG. 13c, after incubation for 24 h, little toxicity of free GVs and PH-GV nanoparticles on cells was detected even in the highest concentration (1 nM). And collapsed GVs also did not induce obvious cytotoxicity to SCC7 cancer cells.

To further investigate the safety of GVs and PH-GVs, the incubation time was prolonged to 48 h in SCC7 cells. FIG. 13d demonstrated that three groups of free GVs, collapsed GVs and PH-GV nanoparticles did not induce significant cell death in a concentration dependent manner. As a result, it was expected that the toxicities of GVs and PH-modified GVs were neglectable in metastatic SCC7 cancer cells.

### Example 13: Biodistribution of GVs and PH-GVs in Nude Mice

Animal experiments were conducted under protocols approved by Animal Care and Use Committee (CC/ACUCC) of Hong Kong Polytechnic University. Suspension of 4×10⁶ SCC7 cells in PBS (80 μL) was injected to subcutaneous sites in athymic nude mice (seven weeks old, female, 20-24 g). When the tumor size (in the right leg region) reached average size of 120 mm³, mice were randomly allocated into three groups, (a) injecting free ICG solution into the tail veins of mice; (b) injecting ICG labelled GVs solution into the tail veins of mice; and (c) injecting ICG labelled PH-GVs solution into the tail veins of mice. Fluorescent imagines were acquired at 0, 0.5, 1, 2, 4, 8, 12, 24 and 48 h after injection using IVIS Lumina II (Caliper Life Sciences, USA; Excitation Filter: 780 nm, Emission Filter: 800 nm).

At the time of highest accumulation after one-dose injection, SCC7 tumors and normal organs (hearts, livers, spleens, kidneys, lungs and muscles) were collected for acquisition of Fluorescent signal intensity.

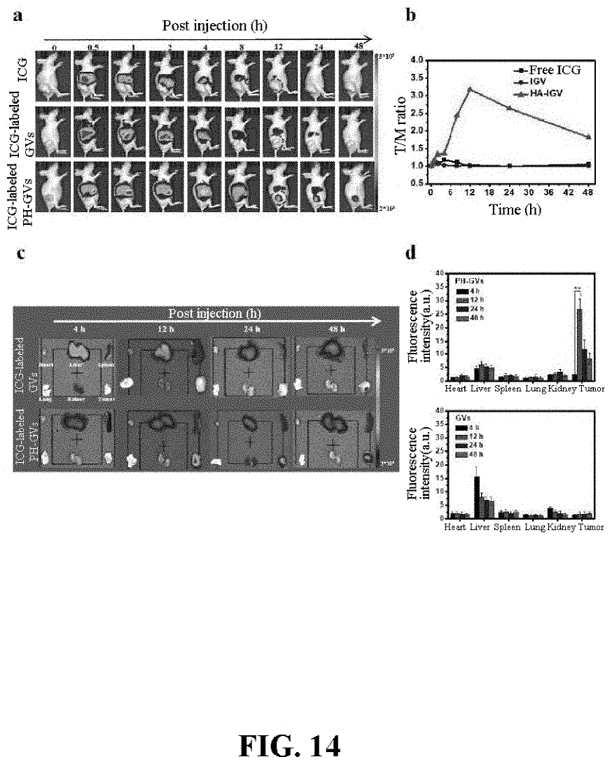

As GVs exhibited superior abilities of targeting and immune escape in vitro, biodistribution and tumor targeting characteristics of the nanoparticles in vivo were further evaluated using a real-time NIRF imaging technique after injection of ICG-labeled PH-GVs into the tumor-bearing mice. As shown in FIGS. 14a-b, for groups of free ICG and ICG-labeled GVs, considerable fluorescence signals were detected in the normal organs (livers, lungs, spleen, etc.) of the mice at the early time points (0.5-2 h), resulting from capture by the reticuloendothelial system (RES) in these organs, and then gradually decreased as a function of time. By contrast, ICG-labeled PH-GVs produced a consistent fluorescence signals in the whole bodies of the mice up to 8 h, without high fluorescence signals increasing in the normal organs. And obvious fluorescence was observed at tumor sites 8 h after intravenous injection. The ICG-labeled PH-GVs fluorescence increased steadily and reached a peak at 12 h. Subsequently, the fluorescence at the tumor site gradually decreased from 24 to 48 h, indicating the sustained excretion of PH-GVs from the tumor tissue. However, no strong fluorescence signal was found in the tumor tissue after administration of ICG and ICG-labeled GVs. This dramatic difference might be ascribed to the enhanced immune escape and specific tumor targeting abilities of PH-GVs.

To investigate whether surface-modified GVs could effectively reduce their liver uptake and improve accumulation in tumor, the targeting ability of the surface-modified GVs in vivo was investigated in SCC7 tumor-bearing model. As shown in FIG. 14c-d, for ICG-labeled GVs-treated group, high fluorescence was detected in the liver at 4 h post injection in vivo. Even at 12 h post treatment, there were still high levels of fluorescence in the liver. Meanwhile, no significant fluorescence appeared in tumors and other organs. The low concentration in tumor and high content in liver indicated most of the GVs were captured by RES, resulting in rapid clearance of nanoparticles in the blood. On the contrary, as for the ICG-labeled PH-GVs-treated mice, the levels of fluorescence in the tumor were much stronger than that of other organs over the course of 48 h. The maximum fluorescent signal in tumor occurred in 12 h post treatment and then slightly decreased with time. On the other hand, low levels of fluorescence were detected in liver and spleen. To sum up, PH modification significantly reduced RES uptake of PH-GVs and improved blood circulation and tumor biodistribution. The excellent tumor accumulating ability of PH-GVs would be beneficial to their further application in US molecular imaging and extensively enhance the therapeutic effect with little side effects.

### Example 14: Cancer US Imaging by PH-GVs In Vivo

Animal experiments were conducted under protocols approved by Animal Care and Use Committee (CC/ACUCC) of Hong Kong Polytechnic University. Suspension of 4×10⁶ SCC7 cells in PBS (80 μL) was injected to subcutaneous sites in nude mice (seven weeks old, female, 20-24 g). When the tumor size (in the right leg region) reached average size of 120 mm³, mice were randomly allocated into two groups, (a) injecting GVs into the tail veins of mice; and (b) injecting PH-GVs into the tail veins of mice. US images of tumor sites were recorded at 0, 0.5, 1, 2, 4, 8, 12, 24 and 48 h after injection using Vevo 2100 imaging system.

At the time of highest accumulation after one-dose injection, a high power of US stimulation was performed, which could induce the collapse of GVs. And the Vevo 2100 imaging system is applied to record the US images and signal intensity before and after US stimulation for analysis. The signal intensities of echo imaging were measured using Vevo 2100 Workstation Software.

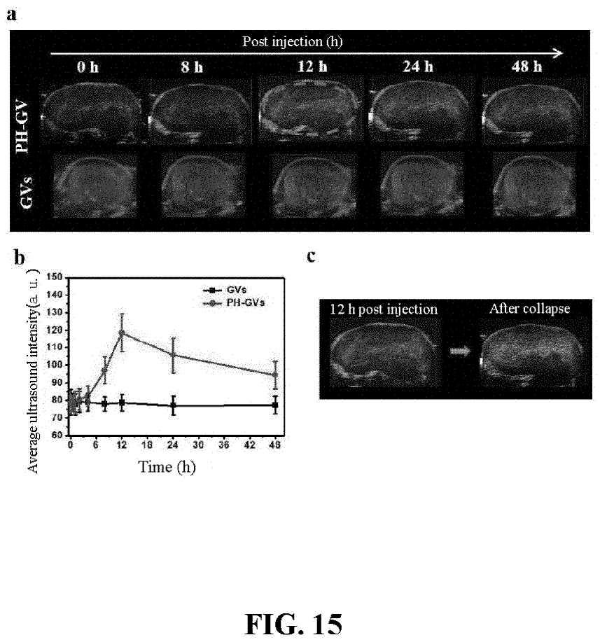

Besides the excellent tumor accumulation of PH-GVs, GVs and PH-GVs were injected intravenously into SCC7 tumor-bearing model to demonstrate that the surface-modified GVs were capable of producing ultrasound contrast in vivo and exhibited robust non-linear signals (FIG. 15a). Firstly, GVs or PH-GVs (200 μL, at the concentration of 20.0 nM) were injected into the tail veins of SCC7 tumor-bearing nude mice, and nonlinear ultrasound images (transmitting at 18 MHz) were acquired using the Vevo 2100 imaging system. The ultrasound images showing tumor sites at 0, 0.5, 1, 2, 4, 8, 12, 24 and 48 h after the injection were presented in FIG. 15a. Green in these images highlighted the region inside the tumor that was enhanced by the ultrasound signal after the intravenous injection. In the PH-GVs group, we could observe dramatically enhanced ultrasound signal intensity in the tumor sites after 8 h. Compared to the signals before PH-GVs injection, US signals in tumor were found increase by 1.05±0.13, 1.2±0.41, 1.51±0.25, and 1.35±0.12 times at 4, 8, 12 and 24 h post injection (p.i.), respectively (FIG. 15b). This accumulation was peaked also at 12 h p.i. as fluorescent imaging demonstrated and started to decrease after 12 h p.i. However, the echo intensity was not found changing significantly in GVs treated mice. To confirm that PH-GVs were the source of the observed contrast, ultrasonic pulses were applied at a supra-collapse pressure (650 kPa), resulting in contrast disappearance (FIG. 15c). Regions of interest containing GVs exhibited 60±14% stronger backscattered signals than buffer-injected controls (p=0.008); this difference disappeared after collapse of GVs (p=0.23).

### Example 15: Retention of PH-GVs in Tumor-Bearing Mice

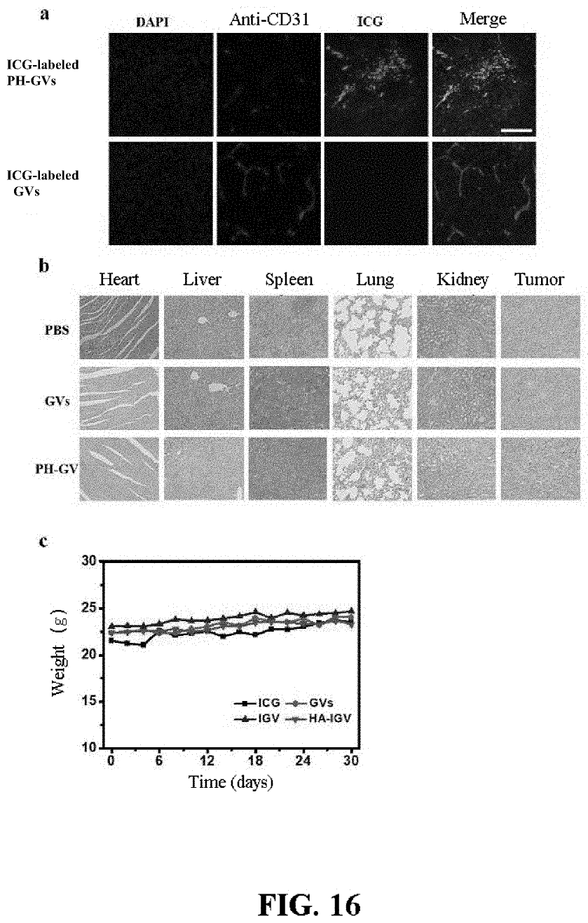

We investigated the interstitial penetration of GVs and PH-GVs inside solid tumors after intravenous injection. Tumor slices extracted from mice at 12 h post intravenous injection of ICG labeled GVs or ICG labeled PH-GVs were stained with DAPI (blue) and anti-CD31 antibody (red) for confocal imaging (FIG. 16a). For ICG labeled GVs group, as shown in FIG. 16a, no ICG fluorescence signals (green) was found in the tumor sections, because the rapid clearance velocity of GVs in vivo resulted in insufficient blood circulation time, and then it was difficult for the free GVs to penetrate through vessels around tumor tissues. In marked contrast, the ICG fluorescence in the PH-GVs groups were much higher than GVs group, indicating that modifying GVs by PH could enhance the tumorous accumulation and retention because of the longer circulation time for EPR effect from PEG and the targetability from HA. More importantly, we observed a great deal of ICG fluorescence signals located far from blood vessels, indicating that PH-GVs nanoparticles might penetrate through the blood vessels surrounding tumor sites and be internalized inside the tumor cells to achieve efficient in-tumor diffusion.

### Example 16: Cytotoxicity of GVs and PH-GVs

The SCC7 cells were seeded in a 96-well plate at a density of 8000 cells per well and cultured for overnight at 37° C. in a 5% CO₂ incubator. The next day, cells were washed with PBS for 3 times and incubated with GVs, collapsed GVs and PH-GV solutions at a series of concentrations for 24 and 48 h under the same condition. Cell viability was evaluated by CCK-8 assay kit. The optical density (OD) was measured at 450 nm and recorded by a microplate reader.

Cytotoxicity was also investigated by 3′6′-bis (O-acetyl)-4′5′-bis[N,N-bis(carboxymethyl)aminomethyl]fluorescein, tetraacetoxymethyl ester (Calcein AM)/Propidium Iodide (PI) staining (Sangon Biotech, Shanghai, China). The U87 cells were seeded in 6-well plate with a density of 1×10⁵ cells and grown to 80-90% confluence. The SCC7 cells were incubated with parallel concentrations of GVs, collapsed GVs and PH-GVs for 24 h. After being washed with PBS for several times and immersed in fresh culture medium (1 mL), the control group in the dark was incubated in fresh DMEM medium. After removing fresh DMEM medium, calcein AM (4 μmol/L) and PI solutions (4 μmol/L) in PBS were added to SCC7 cells and incubated for 30 min at 37° C. with 5% CO₂. Finally, PBS was used to clean the cells three times. Fluorescence images of the cells were obtained by fluorescence microscope.

To investigate the toxicity of GV in vivo, normal organs were harvested and subjected to H&E staining. As shown in FIG. 16b, mice treated with GVs and PH-GVs showed no obvious signal of damage or toxicity from pathologic analysis of hearts, livers, spleens, lungs, and kidneys. As shown in FIG. 16c, the body weight changes in various treatment groups during the period of treatments were observed similar variation trends during 30 days after treatment. Notably, no acute toxicity or adverse effects of GVs and PH-GVs in mice were observed in the present study.

The above description of the embodiments is for the convenience of those of ordinary skill in the art to understand and apply the present invention. It is obvious that those skilled in the art can easily make various modifications to these embodiments, and apply the general principles described herein to other embodiments without creative work. Therefore, the present invention is not limited to the specific embodiments disclosed herein. Any improvements and modifications made by those skilled in the art based on the principles of the present invention, without departing from the scope of the present invention, belong to the protection scope of the present invention.

1. A surface modified targeting gas vesicle (GV) capable of specifically targeting tumor sites, which is surface-modified with a material selected from the group consisting of a targeting biomaterial and a biocompatible material.

2. The targeting GV according to claim 1, wherein the targeting biomaterial is one or more selected from the group consisting of HA, RGD peptide, folic acid, galactose, and glucose; or the biocompatible material is one or more selected from the group consisting of PEG, chitosan, polyurethane, polylactic acid, polyolefin, polysulfone, polycarbonate, and polyacrylonitrile.

3. The targeting GV according to claim 1, wherein the surface of the targeting GV is modified with HA and PEG.

4. A method for preparing the targeting GV of claim 1, comprising:
(a) linking the targeting biomaterial with GV; and
(b) linking the biocompatible material with the product from step (a).

5. A lipid GV surface-modified with a lipid molecule, the lipid molecule is one or more selected from the group consisting of DSPE-PEG, DOPC, PEG, DSPE, PVA, glycerol, glyceride, fatty acid, and phospholipid.

6. The lipid GV according to claim 5, wherein the surface of the lipid GV is modified with DSPE-PEG and DOPC.

7. A gas-filled lipid GV comprising the lipid GV of claim 5 and a therapeutic gas.

8. A lipid targeting GV surface-modified with a lipid molecule and a targeting biomaterial and optionally a biocompatible material:
the lipid molecule is one or more selected from the group consisting of DSPE-PEG, DOPC, PEG, DSPE, PVA, glycerol, glyceride, fatty acid, and phospholipid;
the targeting biomaterial is one or more selected from the group consisting of HA, RGD peptide, folic acid, galactose, and glucose; and
the biocompatible material is one or more selected from the group consisting of PEG, chitosan, polyurethane, polylactic acid, polyolefin, polysulfone, polycarbonate, and polyacrylonitrile.

9. The lipid targeting GV according to claim 8, wherein the surface of the lipid targeting GV is modified with DSPE-PEG, DOPC, and HA, and optionally PEG.

10. A gas-filled lipid targeting GV comprising the lipid targeting GV of claim 8 and a therapeutic gas.

11. A method for preparing the lipid GV of claim 5, comprising:
(a) dissolving the lipid molecule or a mixture of lipid molecules in chloroform, followed by drying;
(b) adding a HEPES buffer to the product from step (a) under agitation to form a cloudy solution; and
(c) adding the product from step (b) to a GV solution to form a lipid GV.

12. A method for preparing the lipid targeting GV of claim 8, comprising:
(a) dissolving the lipid molecule or a mixture of lipid molecules in chloroform, followed by drying;
(b) adding a HEPES buffer to the product from step (a) under agitation to form a cloudy solution; and
(c) linking the targeting biomaterial and GV together, followed by adding the product from step (b) to the GV solution to form a lipid targeting GV.

13. The method according to claim 11, further comprising:
in step (c), filling the GV solution with gas until saturation before adding the product from step (b) to the GV solution, thereby forming the gas-filled lipid GV.

14. A contrast agent or diagnostic agent comprising the targeting GV of claim 1.

15. A therapeutic agent comprising the lipid GV of claim 5.

16. A drug carrier comprising the targeting GV of claim 1 and optionally a drug for treating a disease to be treated.

17. A pharmaceutical composition comprising the therapeutic agent of claim 15.

18. A method for diagnosing a cancer in a subject, the method comprising administering a target GV of claim 1 to the subject.

19. A method for treating a cancer in a subject, the method comprising administering the therapeutic agent of claim 15 to the subject.

20. A method for treating a cancer in a subject, the method comprising:
administering the lipid GV of claim 5 to the subject; and then,
administering a second therapeutic agent or therapy to the subject,
wherein optionally, ultrasound is applied to the subject before administering the second therapeutic agent or therapy to the subject.

21. The method according to claim 19, wherein the cancer is a bladder cancer, a lung cancer, a kidney cancer, a gastric cancer, a colorectal cancer, a liver cancer, a breast cancer, or a melanoma.
